(19) United States
(12) Patent Application Publication
(10) Pub. No.: US 2008/0103164 A1
(43) Pub. Date: May 1, 2008

(54) USEFUL COMPOUNDS FOR HPV INFECTION

(76) Inventors: Kristjan Gudmundsson, Research Triangle Park, NC (US); John Franklin Miller, ResearchTriangle Park, NC (US); Ronald George Sherrill, Research Triangle Park, NC (US); Elizabeth Madalena Turner, Research Triangle Park, NC (US)

Correspondence Address: GLAXOSMITHKLINE;CORPORATE INTELLECTUAL PROPERTY, MAI B475, FIVE MOORE DR., PO BOX 13398, RESEARCH TRIANGLE PARK, NC, 27709-3398, US

(21) Appl. No.: 11/573,111

(22) PCT Filed: Jul. 27, 2005
(86) PCT No.: PCT/US05/26619

§ 371 Date: Feb. 2, 2007

Related U.S. Application Data
(60) Provisional application No. 60/598,102, filed on Aug. 2, 2004.

(51) Int. Cl. A61K 31/437; A61K 31/506; A61P 31/12; A61P 31/20; C07D 471/04

(52) U.S. Cl. 514/275; 514/292; 544/331; 546/85

(57) ABSTRACT

The present invention relates to compounds that are useful in the treatment of human papillomaviruses, and also to the methods for the making and use of such compounds.

### FIELD OF THE INVENTION

The present invention relates to novel compounds that are useful in the treatment of human papillomavirus infections, and also to the methods for the making and use of such compounds.

### BACKGROUND OF THE INVENTION

Human Papillomaviruses (HPVs) are small nonenveloped DNA viruses involved in many conditions and diseases. For example HPVs cause a wide variety of benign and pre-malignant tumors.

HPV is spread by direct contact. HPVs may be divided into two categories: cutaneous and mucosal. The cutaneous HPVs cause warts on hands and feet, such as common, plantar, filiform, or flat warts. The mucosal HPV types infect the anogenital region and the oral cavity. Approximately 100 different types of HPV have been characterized to date. Approximately 40 HPV types specifically infect the genital and oral mucosa. Mucosal HPVs are most frequently sexually transmitted and, with an incidence roughly twice that of herpes simplex virus infection, HPVs are considered one of the most common sexually transmitted diseases (STDs) throughout the world.

Infection with the human papillomavirus (HPV) may not cause any symptoms and does not always produce visible genital warts. When symptoms do develop, they usually occur 2 to 3 months after infection with the virus. Symptoms have been known to develop, however, from 3 weeks to many years after infection occurs. As such, HPV may be spread unknowingly.

More than 25 HPV types that are implicated in anogenital diseases are broadly classified as either low risk or high risk. Low risk HPVs, such as HPV-6 and HPV-11, are the etiological cause of genital warts (condyloma acuminata). High risk HPVs, such as HPV-16, 18, 31, 33, 35, 39, 45, 51, 52, 56, 58, 59, and 68, may not produce visible genital warts. Rather the high-risk viral types may be identified by DNA testing. High risk HPVs such as HPV-16 and HPV-18 may be found on Pap screening tests and be related to precancerous cervical cell change, cervical dysplasia, and cervical cancer. In fact, high-risk HPV types, such as 16, 18, 31, 33, and 35, are strongly associated with precancerous and cancerous changes of the cervix. Most cervical cancers (about 90%). contain one of these high-risk types. High risk HPV infection creates a lifetime risk of invasive cancer in the range of 5-10% for untreated infection.

In addition to cervical cancer, high risk HPVs are associated with a number of anal and perianal cancers.

Current treatments for genital warts and cervical dysplasia include physical removal such as cryotherapy, electrosurgery, and surgical excision. Currently, there are no effective antiviral treatments for HPV infection.

### SUMMARY OF THE INVENTION

One aspect of the present invention includes a method for the treatment or prophylaxis of conditions or disorders due to HPV infection comprising the administration of a compound of formula (I):

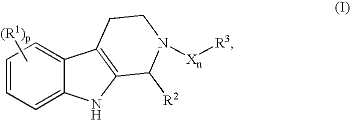

wherein:
each R¹ independently is halogen, haloalkyl, alkyl, alkenyl, alkynyl, cycloalkyl, cycloalkenyl, —R¹⁰cycloalkyl, Ay, AyR⁴, AyOR⁴—NHR¹⁰Ay, Het, HetR⁴, HetOR⁴, —NHHet, —NHR¹⁰Het, —OR⁴, —OAy, —OHet, —R¹⁰OR⁴, —NR⁴R⁵, —NR⁴Ay, —R¹⁰NR⁴R⁵, —R¹⁰NR⁴Ay, —R¹⁰C(O)R⁴, —C(O)R⁴, —CO₂R⁴, —R¹⁰CO₂R⁴, —C(O)NR⁴R⁵, —C(O)Ay, —C(O)NR⁴Ay, —C(O)Het, —C(O)NHR¹⁰Het, —R¹⁰C(O)NR⁴R⁵, —C(S)NR⁴R⁵, —R¹⁰C(S)NR⁴R⁵, —R¹⁰NHC(NH)NR⁴R⁵, —C(NH)NR⁴R⁵, —R¹⁰C(NH)NR⁴R⁵, —S(O)₂NR⁴R⁵, —S(O)₂NR⁴Ay, —R¹⁰SO₂NHCOR⁴, —R¹⁰SO₂NR⁴R⁵, —R¹⁰SO₂R⁴, —S(O)ₘR⁴, cyano, nitro, or azido;
p is 0, 1, 2, 3, or 4;
n is 0 or 1;
m is 0, 1, or 2;

### X is selected from a group consisting of C(O), C(O)O, C(O)Y, S(O), SO₂, S(O)Y, SO₂Y, C(O)OY, C(O)NHY, C(O)YN(H)C(O)OY;

each Y independently is optionally substituted alkylene, optionally substituted cycloalkylene, optionally substituted alkenylene, optionally substituted cycloalkenylene, or optionally substituted alkynylene;
R² is -Ay or -Het, each optionally substituted with one or more halogen, haloalkyl, alkyl, alkenyl, alkynyl, cycloalkyl, cycloalkenyl, —R¹⁰cycloalkyl, Ay, AyR⁴, AyOR⁴—NHR¹⁰Ay, Het, HetR⁴, HetOR⁴, —NHHet, —NHR¹⁰Het, —OR⁴, —OAy, —OHet, —R¹⁰OR⁴, —NR⁴R⁵, —NR⁴Ay, —R¹⁰NR⁴R⁵, —R¹⁰NR⁴Ay, —R¹⁰C(O)R⁴, —C(O)R⁴, —CO₂R⁴, —R¹⁰CO₂R⁴, —C(O)NR⁴R⁵, —C(O)Ay, —C(O)NR⁴Ay, —C(O)Het, —C(O)NHR¹⁰Het, —R¹⁰C(O)NR⁴R⁵, —C(S)NR⁴R⁵, —R¹⁰C(S)NR⁴R⁵, —R¹⁰NHC(NH)NR⁴R⁵, —C(NH)NR⁴R⁵, —R¹⁰C(NH)NR⁴R⁵, —S(O)₂NR⁴R⁵, —S(O)₂NR⁴Ay, —R¹⁰SO₂NHCOR⁴, —R¹⁰SO₂NR⁴R⁵, —R¹⁰SO₂R⁴, —S(O)ₘR⁴, cyano, nitro, or azido;
R³ is alkyl, -Ay or -Het, where Ay or Het may each be optionally substituted with one or more halogen, haloalkyl, alkyl, alkenyl, alkynyl, cycloalkyl, cycloalkenyl, —R¹⁰cycloalkyl, Ay, AyR⁴, AyOR⁴—NHR¹⁰Ay, Het, HetR⁴, HetOR⁴, —NHHet, —NHR¹⁰Het, —OR⁴, —OAy, —OHet, —R¹⁰OR⁴, —NR⁴R⁵, —NR⁴Ay, —R¹⁰NR⁴R⁵, —R¹⁰NR⁴Ay, —R¹⁰C(O)R⁴, —C(O)R⁴, —CO₂R⁴, —R¹⁰CO₂R⁴, —C(O)NR⁴R⁵, —C(O)Ay, —C(O)NR⁴Ay, —C(O)Het, —C(O)NHR¹⁰Het, —R¹⁰C(O)NR⁴R⁵, —C(S)NR⁴R⁵, —R¹⁰C(S)NR⁴R⁵, —R¹⁰NHC(NH)NR⁴R⁵, —C(NH)NR⁴R⁵, —R¹⁰C(NH)NR⁴R⁵, —S(O)₂NR⁴R⁵, —S(O)₂NR⁴Ay, —R¹⁰SO₂NHCOR⁴, —R¹⁰SO₂NR⁴R⁵, —R¹⁰SO₂R⁴, —S(O)ₘR⁴, cyano, nitro, or azido;
each of R⁴ and R⁵ is independently selected from H or alkyl;
each R¹⁰ is an optionally substituted alkylene;

### Ay represents an aryl group;

### Het represents a 5- or 6-membered heterocyclyl or heteroaryl group; the method including administration of pharmaceutically acceptable salts, solvates, and physiologically functional derivatives thereof.

In one embodiment of the present invention, R¹ is selected from halogen, alkyl, cyano, nitro, —OR⁴, Het, —NR⁴R⁵, or —CONR⁴R⁵. Preferably R¹ is halogen or alkyl. Preferably R¹ is F, Cl, Br, or I. More preferably R¹ is Cl or Br.

In one embodiment p is 1 and R¹ is substituted para to the depicted indole nitrogen atom.

In one embodiment R² is Ay. Preferably Ay is phenyl substituted with one or two substituents. Preferably Ay is phenyl optionally substituted with halogen, alkyl, cyano, nitro, —OR⁴, Het, —NR⁴R⁵, or —CONR⁴R⁵. Preferably Ay is phenyl optionally substituted with halogen, alkyl, cyano, —OR⁴, —NR⁴R⁵, or —CONR⁴R⁵.

In one embodiment R² is Het. Preferably Het is dihydrobenzofuran or piperonyl.

In one embodiment n is 1 and X is selected from C(O), C(O)O, C(O)Y, C(O)OY, C(O)NHY, or SO₂Y. Preferably X is selected from C(O)Y, C(O)O, C(O)OY, or C(O)NHY. Preferably, further, R³ is alkyl, Ay, or Het. Preferably R³ is alkyl or Ay.

In one embodiment n is 0 and R³ is Het. Preferably R³ is Het optionally substituted with one or more of halogen, alkyl, cyano, nitro, —OR⁴, Het, Ay, —NR⁴R⁵, or —CONR⁴R⁵.

Particularly preferred compounds include:
Methyl 6-chloro-1-(4-methylphenyl)-1,3,4,9-tetrahydro-2H-β-carboline-2-carboxylate;
6-Chloro-1-(4-methylphenyl)-2-(3-phenylpropanoyl)-2,3,4,9-tetrahydro-1H-β-carboline;
Phenylmethyl 6-chloro-1-(4-methylphenyl)-1,3,4,9-tetrahydro-2H-β-carboline-2-carboxylate;
Methyl 6-fluoro-1-(4-methylphenyl)-1,3,4,9-tetrahydro-2H-β-carboline-2-carboxylate;
6-Fluoro-1-(4-methylphenyl)-2-(3-phenylpropanoyl)-2,3,4,9-tetrahydro-1H-β-carboline;
Phenylmethyl 6-fluoro-1-(4-methylphenyl)-1,3,4,9-tetrahydro-2H-β-carboline-2-carboxylate;
Methyl 6-bromo-1-(4-methylphenyl)-1,3,4,9-tetrahydro-2H-β-carboline-2-carboxylate;
6-Bromo-1-(4-methylphenyl)-2-(3-phenylpropanoyl)-2,3,4,9-tetrahydro-1H-β-carboline;
Phenylmethyl 6-bromo-1-(4-methylphenyl)-1,3,4,9-tetrahydro-2H-β-carboline-2-carboxylate;
(1R)-6-Chloro-1-(4-methylphenyl)-2-(3-phenylpropanoyl)-2,3,4,9-tetrahydro-1H-β-carboline;
(1S)-6-Chloro-1-(4-methylphenyl)-2-(3-phenylpropanoyl)-2,3,4,9-tetrahydro-1H-β-carboline;
1-(4-Methylphenyl)-2-[(phenylmethyl)sulfonyl]-2,3,4,9-tetrahydro-1H-β-carboline;
1-(4-Methylphenyl)-2-(methylsulfonyl)-2,3,4,9-tetrahydro-1H-β-carboline;
(4-{[1-(4-Methylphenyl)-1,3,4,9-tetrahydro-2H-b-carbolin-2-yl]sulfonyl}phenyl)amine.
2-Acetyl-1-(4-methylphenyl)-2,3,4,9-tetrahydro-1H-β-carboline;
1-(4-Methylphenyl)-2-(3-phenylpropanoyl)-2,3,4,9-tetrahydro-1H-β-carboline;
1-(4-Methylphenyl)-2-(3-phenyl-2-propynoyl)-2,3,4,9-tetrahydro-1H-b-carboline;
1-(4-Methylphenyl)-2-[3-(3-pyridinyl)propanoyl]-2,3,4,9-tetrahydro-1H-β-carboline;
Phenylmethyl {2-[1-(4-methylphenyl)-1,3,4,9-tetrahydro-2H-b-carbolin-2-yl]-2-oxoethyl}carbamate;
6-(Methyloxy)-1-(4-methylphenyl)-2-(3-phenylpropanoyl)-2,3,4,9-tetrahydro-1H-β-carboline;
8-Methyl-1-(4-methylphenyl)-2-(3-phenylpropanoyl)-2,3,4,9-tetrahydro-1H-β-carboline;
6-Methyl-1-(4-methylphenyl)-2-(3-phenylpropanoyl)-2,3,4,9-tetrahydro-1H-β-carboline;
7-Fluoro-1-(4-methylphenyl)-2-(3-phenylpropanoyl)-2,3,4,9-tetrahydro-1H-β-carboline;
7-(Methyloxy)-1-(4-methylphenyl)-2-(3-phenylpropanoyl)-2,3,4,9-tetrahydro-1H-β-carboline;
(1R/1S)-1-(2,3-dihydro-1-benzofuran-5-yl)-2-[(2E)-3-phenyl-2-propenoyl]-2,3,4,9-tetrahydro-1H-β-carboline;
(1R)-1-(2,3-dihydro-1-benzofuran-5-yl)-2-[(2E)-3-phenyl-2-propenoyl]-2,3,4,9-tetrahydro-1H-β-carboline;
(1S)-1-(2,3-dihydro-1-benzofuran-5-yl)-2-[(2E)-3-phenyl-2-propenoyl]-2,3,4,9-tetrahydro-1H-β-carboline;
1-(1,3-benzodioxol-5-yl)-2-{4-[4-(methyloxy)phenyl]-2-pyrimidinyl}-2,3,4,9-tetrahydro-1H-β-carboline;
1-(1,3-benzodioxol-5-yl)-2-(2-pyrimidinyl)-2,3,4,9-tetrahydro-1H-β-carboline;
1-(1,3-benzodioxol-5-yl)-2-(4-phenyl-2-pyrimidinyl)-2,3,4,9-tetrahydro-1H-β-carboline;
1-phenyl-2-(4-phenyl-2-pyrimidinyl)-2,3,4,9-tetrahydro-1H-β-carboline;
1-(2,3-dihydro-1-benzofuran-5-yl)-2-(4-phenyl-2-pyrimidinyl)-2,3,4,9-tetrahydro-1H-β-carboline;
1-(2,3-dihydro-1-benzofuran-5-yl)-2-[4-(3-pyridinyl)-2-pyrimidinyl]-2,3,4,9-tetrahydro-1H-β-carboline;
1-(2,3-dihydro-1-benzofuran-5-yl)-2-[4-(1H-imidazol-1-yl)-2-pyrimidinyl]-2,3,4,9-tetrahydro-1H-β-carboline;
1-(2,3-dihydro-1-benzofuran-5-yl)-2-[4-(4-methyl-1H-imidazol-1-yl)-2-pyrimidinyl]-2,3,4,9-tetrahydro-1H-β-carboline;
1-(2,3-dihydro-1-benzofuran-5-yl)-2-[4-(4-methyl-1H-imidazol-1-yl)-2-pyrimidinyl]-2,3,4,9-tetrahydro-1H-β-carboline,
   including pharmaceutically acceptable salts, solvates, and physiologically functional derivatives thereof.

Further, preferred compounds include:
Methyl 6-chloro-1-(4-methylphenyl)-1,3,4,9-tetrahydro-2H-β-carboline-2-carboxylate;
6-Chloro-1-(4-methylphenyl)-2-(3-phenylpropanoyl)-2,3,4,9-tetrahydro-1H-β-carboline;
Phenylmethyl 6-chloro-1-(4-methylphenyl)-1,3,4,9-tetrahydro-2H-β-carboline-2-carboxylate;
Methyl 6-fluoro-1-(4-methylphenyl)-1,3,4,9-tetrahydro-2H-β-carboline-2-carboxylate;
6-Fluoro-1-(4-methylphenyl)-2-(3-phenylpropanoyl)-2,3,4,9-tetrahydro-1H-β-carboline;
Phenylmethyl 6-fluoro-1-(4-methylphenyl)-1,3,4,9-tetrahydro-2H-β-carboline-2-carboxylate;
Methyl 6-bromo-1-(4-methylphenyl)-1,3,4,9-tetrahydro-2H-β-carboline-2-carboxylate;
6-Bromo-1-(4-methylphenyl)-2-(3-phenylpropanoyl)-2,3,4,9-tetrahydro-1H-β-carboline;
Phenylmethyl 6-bromo-1-(4-methylphenyl)-1,3,4,9-tetrahydro-2H-β-carboline-2-carboxylate;
(1S)-6-Chloro-1-(4-methylphenyl)-2-(3-phenylpropanoyl)-2,3,4,9-tetrahydro-1H-β-carboline;
6-(Methyloxy)-1-(4-methylphenyl)-2-(3-phenylpropanoyl)-2,3,4,9-tetrahydro-1H-β-carboline; and
6-Methyl-1-(4-methylphenyl)-2-(3-phenylpropanoyl)-2,3,4,9-tetrahydro-1H-β-carboline, including pharmaceutically acceptable salts, solvates, and physiologically functional derivatives thereof.

Still further, preferred compounds include:
Methyl 6-chloro-1-(4-methylphenyl)-1,3,4,9-tetrahydro-2H-β-carboline-2-carboxylate;
6-Chloro-1-(4-methylphenyl)-2-(3-phenylpropanoyl)-2,3,4,9-tetrahydro-1H-β-carboline;
Phenylmethyl 6-chloro-1-(4-methylphenyl)-1,3,4,9-tetrahydro-2H-β-carboline-2-carboxylate;
Methyl 6-bromo-1-(4-methylphenyl)-1,3,4,9-tetrahydro-2H-β-carboline-2-carboxylate;
6-Bromo-1-(4-methylphenyl)-2-(3-phenylpropanoyl)-2,3,4,9-tetrahydro-1H-β-carboline;
Phenylmethyl 6-bromo-1-(4-methylphenyl)-1,3,4,9-tetrahydro-2H-β-carboline-2-carboxylate; and
(1S)-6-Chloro-1-(4-methylphenyl)-2-(3-phenylpropanoyl)-2,3,4,9-tetrahydro-1H-β-carboline,
   including pharmaceutically acceptable salts, solvates, and physiologically functional derivatives thereof.

One aspect of the present invention includes the present method for the treatment or prophylaxis of diseases and conditions caused by oncogenic viruses, including adenoviruses, retroviruses, and viruses from the papovavirus family, including polyoma viruses and papilloma viruses. Preferably the condition or disease is warts, genital warts, cervical dysplasia, recurrent respiratory papillomatosis, or cancers associated with papillomavirus infection. Particularly the cancer is anogenital cancers, head and neck cancers, and skin cancers. More particularly the anogenital cancers are cervical, anal and perianal, vulvar, vaginal, and penile cancers; the head and neck cancers are oral pharyngeal region and esophagus cancers; and the skin cancers are basal cell carcinoma and squamous cell carcinoma.

Another aspect of the present invention includes the use of a compound of formula (I):

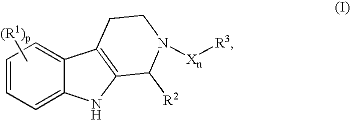

wherein:
each R¹ independently is halogen, haloalkyl, alkyl, alkenyl, alkynyl, cycloalkyl, cycloalkenyl, —R¹⁰cycloalkyl, Ay, AyR⁴, AyOR⁴—NHR¹⁰Ay, Het, HetR⁴, HetOR⁴, —NHHet, —NHR¹⁰Het, —OR⁴, —OAy, —OHet, —R¹⁰OR⁴, —NR⁴R⁵, —NR⁴Ay, —R¹⁰NR⁴R⁵, —R¹⁰NR⁴Ay —R¹⁰C(O)R⁴, —C(O)R⁴, —CO₂R⁴, —R¹⁰CO₂R⁴, —C(O)NR⁴R⁵, —C(O)Ay, —C(O)NR⁴Ay, —C(O)Het, —C(O)NHR¹⁰Het, —R¹⁰C(O)NR⁴R⁵, —C(S)NR⁴R⁵, —R¹⁰C(S)NR⁴R⁵, —R¹⁰NHC(NH)NR⁴R⁵, —C(NH)NR⁴R⁵, —R¹⁰C(NH)NR⁴R⁵, —S(O)₂NR⁴R⁵, —S(O)₂NR⁴Ay, —R¹⁰SO₂NHCOR⁴, —R¹⁰SO₂NR⁴R⁵, —R¹⁰SO₂R⁴, —S(O)ₘR⁴, cyano, nitro, or azido;
p is 0, 1, 2, 3, or 4;
n is 0 or 1;
m is 0, 1, or 2;

### X is selected from a group consisting of C(O), C(O)O, C(O)Y, S(O), SO₂, S(O)Y, SO₂Y, C(O)OY, C(O)NHY, C(O)YN(H)C(O)OY;

each Y independently is optionally substituted alkylene, optionally substituted cycloalkylene, optionally substituted alkenylene, optionally substituted cycloalkenylene, or optionally substituted alkynylene;
R² is -Ay or -Het, each optionally substituted with one or more halogen, haloalkyl, alkyl, alkenyl, alkynyl, cycloalkyl, cycloalkenyl, —R¹⁰cycloalkyl, Ay, AyR⁴, AyOR⁴—NHR¹⁰Ay, Het, HetR⁴, HetOR⁴, —NHHet, —NHR¹⁰Het, —OR⁴, —OAy, —OHet, —R¹⁰OR⁴, —NR⁴R⁵, —NR⁴Ay, —R¹⁰NR⁴R⁵, —R¹⁰NR⁴Ay, —R¹⁰C(O)R⁴, —C(O)R⁴, —CO₂R⁴, —R¹⁰CO₂R⁴, —C(O)NR⁴R⁵, —C(O)Ay, —C(O)NR⁴Ay, —C(O)Het, —C(O)NHR¹⁰Het, —R¹⁰C(O)NR⁴R⁵, —C(S)NR⁴R⁵, —R¹⁰C(S)NR⁴R⁵, —R¹⁰NHC(NH)NR⁴R⁵, —C(NH)NR⁴R⁵, —R¹⁰C(NH)NR⁴R⁵, —S(O)₂NR⁴R⁵, —S(O)₂NR⁴Ay, —R¹⁰SO₂NHCOR⁴, —R¹⁰SO₂NR⁴R⁵, —R¹⁰SO₂R⁴, —S(O)ₘR⁴, cyano, nitro, or azido;
R³ is alkyl, -Ay or -Het, where Ay or Het may each be optionally substituted with one or more halogen, haloalkyl, alkyl, alkenyl, alkynyl, cycloalkyl, cycloalkenyl, —R¹⁰cycloalkyl, Ay, AyR⁴, AyOR⁴—NHR¹⁰Ay, Het, HetR⁴, HetOR⁴, —NHHet, —NHR¹⁰Het, —OR⁴, —OAy, —OHet, —R¹⁰OR⁴, —NR⁴R⁵, —NR⁴Ay, —R¹⁰NR⁴R⁵, —R¹⁰NR⁴Ay, —R¹⁰C(O)R⁴, —C(O)R⁴, —CO₂R⁴, —R¹⁰CO₂R⁴, —C(O)NR⁴R⁵, —C(O)Ay, —C(O)NR⁴Ay, —C(O)Het, —C(O)NHR¹⁰Het, —R¹⁰C(O)NR⁴R⁵, —C(S)NR⁴R⁵, —R¹⁰C(S)NR⁴R⁵, —R¹⁰NHC(NH)NR⁴R⁵, —C(NH)NR⁴R⁵, —R¹⁰C(NH)NR⁴R⁵, —S(O)₂NR⁴R⁵, —S(O)₂NR⁴Ay, —R¹⁰SO₂NHCOR⁴, —R¹⁰SO₂NR⁴R⁵, —R¹⁰SO₂R⁴, —S(O)ₘR⁴, cyano, nitro, or azido;
each of R⁴ and R⁵ is independently selected from H or alkyl;
each R¹⁰ is an optionally substituted alkylene;

### Ay represents an aryl group;

### Het represents a 5- or 6-membered heterocyclyl or heteroaryl group;

including pharmaceutically acceptable salts, solvates, and physiologically functional derivatives thereof, in the manufacture of a medicament for use in the treatment or prophylaxis of oncogenic viruses, including adenoviruses, retroviruses, and viruses from the papovavirus family, including polyoma viruses and papilloma viruses.

Particularly the use relates to the treatment or prophylaxis of conditions or disorders due to HPV infection. More particularly the use relates to warts, genital warts, cervical dysplasia, recurrent respiratory papillomatosis, or cancers associated with papillomavirus infection. Particularly the cancer is anogenital cancers, head and neck cancers, and skin cancers. More particularly the anogenital cancers are cervical, anal and perianal, vulvar, vaginal, and penile cancers; the head and neck cancers are oral pharyngeal region and esophagus cancers; and the skin cancers are basal cell carcinoma and squamous cell carcinoma.

### DETAILED DESCRIPTION OF THE PREFERRED EMBODIMENT

Terms are used within their accepted meanings. The following definitions are meant to clarify, but not limit, the terms defined.

As used herein the term “alkyl” refers to a straight or branched chain hydrocarbon, preferably having from one to twelve carbon atoms, which may be optionally substituted. Examples of “alkyl” as used herein include, but are not limited to, methyl, ethyl, propyl, isopropyl, isobutyl, n-butyl, tert-butyl, isopentyl, n-pentyl, and the like.

As used throughout this specification, the preferred number of atoms, such as carbon atoms, will be represented by, for example, the phrase “Cₓ₋C_{y} alkyl,” which refers to an alkyl group, as herein defined, containing the specified number of carbon atoms. Similar terminology will apply for other preferred terms and ranges as well.

As used herein the term “alkenyl” refers to a straight or branched chain aliphatic hydrocarbon containing one or more carbon-to-carbon double bonds that may be optionally substituted. Examples include, but are not limited to, vinyl, allyl, and the like.

As used herein the term “alkynyl” refers to a straight or branched chain aliphatic hydrocarbon containing one or more carbon-to-carbon triple bonds that may be optionally substituted. Examples include, but are not limited to, ethynyl and the like.

As used herein, the term “alkylene” refers to a straight or branched chain divalent hydrocarbon radical, preferably having from one to ten carbon atoms. Alkylene groups as defined herein may optionally be substituted. Examples of “alkylene” as used herein include, but are not limited to, methylene, ethylene, n-propylene, n-butylene, and the like.

As used herein, the term “alkenylene” refers to a straight or branched chain divalent hydrocarbon radical, preferably having from one to ten carbon atoms, containing one or more carbon-to-carbon double bonds that may be optionally substituted. Examples include, but are not limited to, vinylene, allylene or 2-propenylene, and the like.

As used herein, the term “alkynylene” refers to a straight or branched chain divalent hydrocarbon radical, preferably having from one to ten carbon atoms, containing one or more carbon-to-carbon triple bonds that may be optionally substituted. Examples include, but are not limited to, ethynylene and the like.

As used herein, the term “cycloalkyl” refers to an optionally substituted non-aromatic cyclic hydrocarbon ring, which optionally includes an alkylene linker through which the cycloalkyl may be attached. Exemplary “cycloalkyl” groups include, but are not limited to, cyclopropyl, cyclobutyl, cyclopentyl, cyclohexyl, cycloheptyl, and the like. As used herein, the term “cycloalkyl” includes an optionally substituted fused polycyclic hydrocarbon saturated ring and aromatic ring system, namely polycyclic hydrocarbons with less than maximum number of non-cumulative double bonds, for example where a saturated hydrocarbon ring (such as a cyclopentyl ring) is fused with an aromatic ring (herein “aryl,” such as a benzene ring) to form, for example, groups such as indane.

As used herein, the term “cycloalkenyl” refers to an optionally substituted non-aromatic cyclic hydrocarbon ring containing one or more carbon-to-carbon double bonds which optionally includes an alkylene linker through which the cycloalkenyl may be attached. Exemplary “cycloalkenyl” groups include, but are not limited to, cyclopropenyl, cyclobutenyl, cyclopentenyl, cyclohexenyl, cycloheptenyl, and the like.

As used herein, the term “cycloalkylene” refers to a divalent, optionally substituted non-aromatic cyclic hydrocarbon ring. Exemplary “cycloalkylene” groups include, but are not limited to, cyclopropylene, cyclobutylene, cyclopentylene, cyclohexylene, cycloheptylene, including fused systems with reference to the above definition for cycloalkyl.

As used herein, the term “cycloalkenylene” refers to a divalent optionally substituted non-aromatic cyclic hydrocarbon ring containing one or more carbon-to-carbon double bonds. Exemplary “cycloalkenylene” groups include, but are not limited to, cyclopropenylene, cyclobutenylene, cyclopentenylene, cyclohexenylene, cycloheptenylene, and the like.

As used herein, the term “heterocycle” or “heterocyclyl” refers to an optionally substituted mono- or polycyclic ring system containing one or more degrees of unsaturation and also containing one or more heteroatoms. Preferred heteroatoms include N, O, and/or S, including N-oxides, sulfur oxides, and dioxides. Preferably the ring is three to twelve-membered and is either fully saturated or has one or more degrees of unsaturation. Such rings may be optionally fused to one or more of another “heterocyclic” ring(s) or cycloalkyl ring(s). Examples of “heterocyclic” groups include, but are not limited to, tetrahydrofuran, pyran, 1,4-dioxane, 1,3-dioxane, piperidine, pyrrolidine, morpholine, tetrahydrothiopyran, and tetrahydrothiophene.

As used herein, the term “aryl” refers to an optionally substituted benzene ring or to an optionally substituted fused benzene ring system, for example anthracene, phenanthrene, or naphthalene ring systems. Examples of “aryl” groups include, but are not limited to, phenyl, 2-naphthyl, and 1-naphthyl.

As used herein, the term “heteroaryl” refers to an optionally substituted monocyclic five to seven membered aromatic ring, or to an optionally substituted fused bicyclic aromatic ring system comprising two of such aromatic rings. These heteroaryl rings contain one or more nitrogen, sulfur, and/or oxygen atoms, where N-oxides, sulfur oxides, and dioxides are permissible heteroatom substitutions. Additionally, the term is meant to encompass heterocycles fused with aromatic ring systems. Examples of “heteroaryl” groups used herein include, but should not be limited to, furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, dihydrobenzofuran, piperonyl (benzodioxolyl), benzothiophene, indole, indazole, benzimidizole, imidazopyridine, pyrazolopyridine, pyrazolopyrimidine, and substituted versions thereof.

As used herein the term “halogen” refers to fluorine, chlorine, bromine, or iodine.

As used herein the term “haloalkyl” refers to an alkyl group, as defined herein, which is substituted with at least one halogen. Examples of branched or straight chained “haloalkyl” groups useful in the present invention include, but are not limited to, methyl, ethyl, propyl, isopropyl, n-butyl, and t-butyl substituted independently with one or more halogens, e.g., fluoro, chloro, bromo, and iodo. The term “haloalkyl” should be interpreted to include such substituents as perfluoroalkyl groups and the like.

As used herein the term “alkoxy” refers to the group —OR, where R is alkyl as defined above.

As used herein the term “alkoxycarbonyl” refers to groups such as:

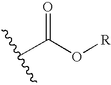

where the R represents an alkyl group as herein defined.

As used herein the term “aryloxycarbonyl” refers to groups such as:

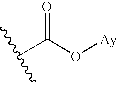

where the Ay represents an aryl group as herein defined.

As used herein the term “heteroaryloxycarbonyl” refers to groups such as:

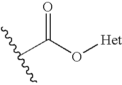

where the Het represents a heteroaryl group as herein defined.

As used herein the term “nitro” refers to the group —NO₂.

As used herein the term “cyano” refers to the group —CN.

As used herein the term “azido” refers to the group —N₃.

As used herein the term “acyl” refers to the group RC(O)—, where R is alkyl, aryl, heteroaryl, or heterocyclyl, as each is defined herein.

As used herein the term “oxo” refers to the group ═O.

As used herein throughout the present specification, the phrase “optionally substituted” or variations thereof denote an optional substitution, including multiple degrees of substitution, with one or more substituent group. The phrase should not be interpreted so as to be imprecise or duplicative of substitution patterns herein described or depicted. Rather, those of ordinary skill in the art will appreciate that the phrase is included to provide for obvious modifications, which are encompassed within the scope of the appended claims.

The compounds of formulas (I) may crystallize in more than one form, a characteristic known as polymorphism, and such polymorphic forms (“polymorphs”) are within the scope of formula (I). Polymorphism generally can occur as a response to changes in temperature, pressure, or both. Polymorphism can also result from variations in the crystallization process. Polymorphs can be distinguished by various physical characteristics known in the art such as x-ray diffraction patterns, solubility, and melting point.

Certain of the compounds described herein contain one or more chiral centers, or may otherwise be capable of existing as multiple stereoisomers. The scope of the present invention includes mixtures of stereoisomers as well as purified enantiomers or enantiomerically/diastereomerically enriched mixtures. Also included within the scope of the invention are the individual isomers of the compounds represented by formula (I), as well as any wholly or partially equilibrated mixtures thereof. The present invention also includes the individual isomers of the compounds represented by the formulas above as mixtures with isomers thereof in which one or more chiral centers are inverted. Typically, but not absolutely, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts.

Representative pharmaceutically acceptable salts include, but should not be considered limited to, acetate, benzenesulfonate, benzoate, bicarbonate, bisulfate, bitartrate, borate, calcium edetate, camsylate, carbonate, clavulanate, citrate, dihydrochloride, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylsulfate, monopotassium maleate, mucate, napsylate, nitrate, N-methylglucamine, oxalate, pamoate (embonate), palmitate, pantothenate, phosphate/diphosphate, polygalacturonate, potassium, salicylate, sodium, stearate, subacetate, succinate, sulfate, tannate, tartrate, teoclate, tosylate, triethiodide, trimethylammonium, and valerate salts. Other salts, which are not pharmaceutically acceptable, may be useful in the preparation of compounds of this invention and these should be considered to form a further aspect of the invention.

As used herein, the term “solvate” refers to a complex of variable stoichiometry formed by a solute (in this invention, a compound of Formula I, or a salt or physiologically functional derivative thereof) and a solvent. Such solvents, for the purpose of the invention, should not interfere with the biological activity of the solute. Non-limiting examples of suitable solvents include, but are not limited to water, methanol, ethanol, and acetic acid. Preferably the solvent used is a pharmaceutically acceptable solvent. Non-limiting examples of suitable pharmaceutically acceptable solvents include water, ethanol, and acetic acid. Most preferably the solvent used is water.

As used herein, the term “physiologically functional derivative” refers to any pharmaceutically acceptable derivative of a compound of the present invention that, upon administration to a mammal, is capable of providing (directly or indirectly) a compound of the present invention or an active metabolite thereof. Such derivatives, for example, esters and amides, will be clear to those skilled in the art, without undue experimentation. Reference may be made to the teaching of Burger's Medicinal Chemistry And Drug Discovery, 5^{th} Edition, Vol 1: Principles and Practice, which is incorporated herein by reference to the extent that it teaches physiologically functional derivatives.

As used herein, the term “effective amount” means that amount of a drug or pharmaceutical agent that will elicit the biological or medical response of a tissue, system, animal, or human that is being sought, for instance, by a researcher or clinician. The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. For use in therapy, therapeutically effective amounts of a compound of formula (I), as well as salts, solvates, and physiological functional derivatives thereof, may be administered as the raw chemical. Additionally, the active ingredient may be presented as a pharmaceutical composition.

Accordingly, the invention further provides pharmaceutical compositions that include effective amounts of compounds of the formula (I) and salts, solvates, and physiological functional derivatives thereof, and one or more pharmaceutically acceptable carriers, diluents, or excipients. The compounds of formula (I) and salts, solvates, and physiologically functional derivatives thereof, are as herein described. The carrier(s), diluent(s) or excipient(s) must be acceptable, in the sense of being compatible with the other ingredients of the formulation and not deleterious to the recipient of the pharmaceutical composition.

In accordance with another aspect of the invention there is also provided a process for the preparation of a pharmaceutical formulation including admixing a compound of the formula (I) or salts, solvates, and physiological functional derivatives thereof, with one or more pharmaceutically acceptable carriers, diluents or excipients.

A therapeutically effective amount of a compound of the present invention will depend upon a number of factors. For example, the species, age, and weight of the recipient, the precise condition requiring treatment and its severity, the nature of the formulation, and the route of administration are all factors to be considered. The therapeutically effective amount ultimately should be at the discretion of the attendant physician or veterinarian. Regardless, an effective amount of a compound of formula (I) for the treatment of humans suffering from frailty, generally, should be in the range of 0.1 to 100 mg/kg body weight of recipient (mammal) per day. More usually the effective amount should be in the range of 1 to 10 mg/kg body weight per day. Thus, for a 70 kg adult mammal the actual amount per day would usually be from 70 to 700 mg. This amount may be given in a single dose per day or in a number (such as two, three, four, five, or more) of sub-doses per day such that the total daily dose is the same. An effective amount of a salt, solvate, or physiologically functional derivative thereof, may be determined as a proportion of the effective amount of the compound of formula (I) per se. Similar dosages should be appropriate for treatment of the other conditions referred to herein.

Pharmaceutical formulations may be presented in unit dose forms containing a predetermined amount of active ingredient per unit dose. Such a unit may contain, as a non-limiting example, 0.5 mg to 1 g of a compound of the formula (I), depending on the condition being treated, the route of administration, and the age, weight, and condition of the patient. Preferred unit dosage formulations are those containing a daily dose or sub-dose, as herein above recited, or an appropriate fraction thereof, of an active ingredient. Such pharmaceutical formulations may be prepared by any of the methods well known in the pharmacy art.

Pharmaceutical formulations may be adapted for administration by any appropriate route, for example by an oral (including buccal or sublingual), rectal, nasal, topical (including buccal, sublingual or transdermal), vaginal, or parenteral (including subcutaneous, intramuscular, intravenous or intradermal) route. Such formulations may be prepared by any method known in the art of pharmacy, for example by bringing into association the active ingredient with the carrier(s) or excipient(s). By way of example, and not meant to limit the invention, with regard to certain conditions and disorders for which the compounds of the present invention are believed useful certain routes will be preferable to others. Based upon the physical manifestations that are often associated with HPV infection, rectal, topical, or vaginal routes of administration may be preferable. As one example, for the treatment or prophylaxis of cervical dysplasia the preferred route may be a vaginal route.

Pharmaceutical formulations adapted for oral administration may be presented as discrete units such as capsules or tablets; powders or granules; solutions or suspensions, each with aqueous or non-aqueous liquids; edible foams or whips; or oil-in-water liquid emulsions or water-in-oil liquid emulsions. For instance, for oral administration in the form of a tablet or capsule, the active drug component can be combined with an oral, non-toxic pharmaceutically acceptable inert carrier such as ethanol, glycerol, water, and the like. Generally, powders are prepared by comminuting the compound to a suitable fine size and mixing with an appropriate pharmaceutical carrier such as an edible carbohydrate, as, for example, starch or mannitol. Flavorings, preservatives, dispersing agents, and coloring agents can also be present.

Capsules are made by preparing a powder, liquid, or suspension mixture and encapsulating with gelatin or some other appropriate shell material. Glidants and lubricants such as colloidal silica, talc, magnesium stearate, calcium stearate, or solid polyethylene glycol can be added to the mixture before the encapsulation. A disintegrating or solubilizing agent such as agar-agar, calcium carbonate or sodium carbonate can also be added to improve the availability of the medicament when the capsule is ingested. Moreover, when desired or necessary, suitable binders, lubricants, disintegrating agents, and coloring agents can also be incorporated into the mixture. Examples of suitable binders include starch, gelatin, natural sugars such as glucose or beta-lactose, corn sweeteners, natural and synthetic gums such as acacia, tragacanth, or sodium alginate, carboxymethylcellulose, polyethylene glycol, waxes, and the like. Lubricants useful in these dosage forms include, for example, sodium oleate, sodium stearate, magnesium stearate, sodium benzoate, sodium acetate, sodium chloride, and the like. Disintegrators include, without limitation, starch, methyl cellulose, agar, bentonite, xanthan gum, and the like.

Tablets are formulated, for example, by preparing a powder mixture, granulating or slugging, adding a lubricant and disintegrant, and pressing into tablets. A powder mixture may be prepared by mixing the compound, suitably comminuted, with a diluent or base as described above. Optional ingredients include binders such as carboxymethylcellulose, aliginates, gelatins, or polyvinyl pyrrolidone, solution retardants such as paraffin, resorption accelerators such as a quaternary salt, and/or absorption agents such as bentonite, kaolin, or dicalcium phosphate. The powder mixture can be wet-granulated with a binder such as syrup, starch paste, acadia mucilage or solutions of cellulosic or polymeric materials, and forcing through a screen. As an alternative to granulating, the powder mixture can be run through the tablet machine and the result is imperfectly formed slugs broken into granules. The granules can be lubricated to prevent sticking to the tablet-forming dies by means of the addition of stearic acid, a stearate salt, talc or mineral oil. The lubricated mixture is then compressed into tablets. The compounds of the present invention can also be combined with a free flowing inert carrier and compressed into tablets directly without going through the granulating or slugging steps. A clear or opaque protective coating consisting of a sealing coat of shellac, a coating of sugar or polymeric material, and a polish coating of wax can be provided. Dyestuffs can be added to these coatings to distinguish different unit dosages.

Oral fluids such as solutions, syrups, and elixirs can be prepared in dosage unit form so that a given quantity contains a predetermined amount of the compound. Syrups can be prepared, for example, by dissolving the compound in a suitably flavored aqueous solution, while elixirs are prepared through the use of a non-toxic alcoholic vehicle. Suspensions can be formulated generally by dispersing the compound in a non-toxic vehicle. Solubilizers and emulsifiers such as ethoxylated isostearyl alcohols and polyoxy ethylene sorbitol ethers, preservatives; flavor additives such as peppermint oil, or natural sweeteners, saccharin, or other artificial sweeteners; and the like can also be added.

Where appropriate, dosage unit formulations for oral administration can be microencapsulated. The formulation can also be prepared to prolong or sustain the release as for example by coating or embedding particulate material in polymers, wax or the like.

The compounds of formula (I) and salts, solvates, and physiological functional derivatives thereof, can also be administered in the form of liposome delivery systems, such as small unilamellar vesicles, large unilamellar vesicles, and multilamellar vesicles. Liposomes can be formed from a variety of phospholipids, such as cholesterol, stearylamine, or phosphatidylcholines.

The compounds of formula (I) and salts, solvates, and physiologically functional derivatives thereof may also be delivered by the use of monoclonal antibodies as individual carriers to which the compound molecules are coupled.

The compounds may also be coupled with soluble polymers as targetable drug carriers. Such polymers can include polyvinylpyrrolidone (PVP), pyran copolymer, polyhydroxypropylmethacrylamide-phenol, polyhydroxyethyl-aspartamidephenol, or polyethyleneoxidepolylysine substituted with palmitoyl residues. Furthermore, the compounds may be coupled to a class of biodegradable polymers useful in achieving controlled release of a drug; for example, polylactic acid, polyepsilon caprolactone, polyhydroxy butyric acid, polyorthoesters, polyacetals, polydihydropyrans, polycyanoacrylates, and cross-linked or amphipathic block copolymers of hydrogels.

Pharmaceutical formulations adapted for transdermal administration may be presented as discrete patches intended to remain in intimate contact with the epidermis of the recipient for a prolonged period of time. For example, the active ingredient may be delivered from the patch by iontophoresis as generally described in Pharmaceutical Research, 3(6), 318 (1986), incorporated herein by reference as related to such delivery systems.

Pharmaceutical formulations adapted for topical administration may be formulated as ointments, creams, suspensions, lotions, powders, solutions, pastes, gels, sprays, aerosols, or oils.

For treatments of the eye or other external tissues, for example mouth and skin, the formulations may be applied as a topical ointment or cream. When formulated in an ointment, the active ingredient may be employed with either a paraffinic or a water-miscible ointment base. Alternatively, the active ingredient may be formulated in a cream with an oil-in-water cream base or a water-in-oil base.

Pharmaceutical formulations adapted for topical administrations to the eye include eye drops wherein the active ingredient is dissolved or suspended in a suitable carrier, especially an aqueous solvent.

Pharmaceutical formulations adapted for topical administration in the mouth include lozenges, pastilles, and mouthwashes.

Pharmaceutical formulations adapted for nasal administration, where the carrier is a solid, include a coarse powder having a particle size for example in the range 20 to 500 microns. The powder is administered in the manner in which snuff is taken, i.e., by rapid inhalation through the nasal passage from a container of the powder held close up to the nose. Suitable formulations wherein the carrier is a liquid, for administration as a nasal spray or as nasal drops, include aqueous or oil solutions of the active ingredient.

Pharmaceutical formulations adapted for administration by inhalation include fine particle dusts or mists, which may be generated by means of various types of metered dose pressurized aerosols, nebulizers, or insufflators.

Pharmaceutical formulations adapted for rectal administration may be presented as suppositories or as enemas.

Pharmaceutical formulations adapted for vaginal administration may be presented as pessaries, tampons, creams, gels, pastes, foams, or spray formulations.

Pharmaceutical formulations adapted for parenteral administration include aqueous and non-aqueous sterile injection solutions which may contain anti-oxidants, buffers, bacteriostats, and solutes that render the formulation isotonic with the blood of the intended recipient; and aqueous and non-aqueous sterile suspensions which may include suspending agents and thickening agents. The formulations may be presented in unit-dose or multi-dose containers, for example sealed ampules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets.

In addition to the ingredients particularly mentioned above, the formulations may include other agents conventional in the art having regard to the type of formulation in question. For example, formulations suitable for oral administration may include flavoring or coloring agents.

The compounds of the present invention and their salts, solvates, and physiologically functional derivatives thereof, may be employed alone or in combination with other therapeutic agents. The compound(s) of formula (I) and the other pharmaceutically active agent(s) may be administered together or separately and, when administered separately, administration may occur simultaneously or sequentially, in any order. The amounts of the compound(s) of formula (I) and the other pharmaceutically active agent(s) and the relative timings of administration will be selected in order to achieve the desired combined therapeutic effect. The administration in combination of a compound of formula (I) salts, solvates, or physiologically functional derivatives thereof with other treatment agents may be in combination by administration concomitantly in: (1) a unitary pharmaceutical composition including both compounds; or (2) separate pharmaceutical compositions each including one of the compounds. Alternatively, the combination may be administered separately in a sequential manner wherein one treatment agent is administered first and the other second or vice versa. Such sequential administration may be close in time or remote in time.

The compounds of the present invention may be used in the treatment of a variety of disorders and conditions and, as such, the compounds of the present invention may be used in combination with a variety of other suitable therapeutic agents useful in the treatment or prophylaxis of those disorders or conditions. Treatment will depend upon the nature and type of HPV infection. As discussed briefly above, treatment for warts can be divided into ablative and medical approaches. The compounds of the present invention may be combined with either or both approaches.

Ablative methods include classic surgical excision and destruction by electrodesiccation, laser, or liquid nitrogen. Thus, the compounds of the present invention may be used in conjunction with such methods or upon reoccurrence after such methods. The compounds of the present invention may be used in conjunction with ablative methods to reduce the frequency of reoccurrence.

Alternatively, the present invention may be combined with other medical therapies including a variety of cytotoxic or antiviral agents. For example, and not meant to limit the invention, the compounds of the present invention may be combined with other therapeutic agents such as 5-fluorouracil, retinoic acid, podophyllin, podofilox, keratolytic agents such as salicylic acid and/or lactic acid, haptens such as diphencyprone (DPC), squaric acid dibutyl ester (SADBE) or dinitrochlorobenzene (DNCB), formalin, topical trichloroacetic acid, topical tretinoin, cidofovir, imiquimod and/or cytokines such as interferon alfa-2b.

One aspect of the present invention is the use of the compounds of the present invention for the treatment or prophylaxis of a variety of disorders including, but not limited to, diseases and conditions caused by oncogenic viruses, including adenoviruses, retroviruses, and viruses from the papovavirus family, such as polyoma viruses and papilloma viruses and more particularly papilloma viral infections. The present invention includes administering to a subject in need thereof a therapeutically effective amount of a compound of the present invention or a pharmaceutically acceptable salt, solvate or physiologically functional derivative thereof.

More specifically, the present invention includes the treatment or prophylaxis of conditions or diseases associated with papilloma viral infections. These conditions and diseases include warts (e.g. plantar warts), genital warts, recurrent respiratory papillomatosis (e.g., laryngeal papillomas), and cancers associated with papillomavirus infection. Cancers that have been associated with papillomavirus infection include anogenital cancers (e.g., cervical, anal and perianal, vulvar, vaginal, penile cancers), head and neck cancers (e.g., oral pharyngeal region, esophagus), and skin cancers (e.g., basal cell carcinoma, squamous cell carcinoma). The present invention includes administering to a subject in need thereof a therapeutically effective amount of a compound of the present invention or a salt, solvate or physiologically functional derivative thereof.

The compounds of this invention may be made by a variety of methods, including well-known standard synthetic methods. Illustrative general synthetic methods are set out below and then specific compounds of the invention are prepared in the working Examples.

In all of the examples described below, protecting groups for sensitive or reactive groups are employed where necessary in accordance with general principles of synthetic chemistry. Protecting groups are manipulated according to standard methods of organic synthesis (T. W. Green and P. G. M. Wuts (1991) Protecting Groups in Organic Synthesis, John Wiley & Sons, incorporated by reference with regard to protecting groups). These groups are removed at a convenient stage of the compound synthesis using methods that are readily apparent to those skilled in the art. The selection of processes as well as the reaction conditions and order of their execution shall be consistent with the preparation of compounds of formula (I).

Those skilled in the art will recognize if a stereocenter exists in compounds of formula (I). Accordingly, the present invention includes all possible stereoisomers and includes not only racemic compounds but the individual enantiomers as well. When a compound is desired as a single enantiomer, such may be obtained by stereospecific synthesis, by resolution of the final product or any convenient intermediate, or by chiral chromatographic methods as are known in the art. Resolution of the final product, an intermediate, or a starting material may be effected by any suitable method known in the art. See, for example, Stereochemistry of Organic Compounds by E. L. Eliel, S. H. Wilen, and L. N. Mander (Wiley-Interscience, 1994), incorporated by reference with regard to stereochemistry.

### EXPERIMENTAL SECTION

As used herein the symbols and conventions used in these processes, schemes and examples, regardless of whether a particular abbreviation is specifically defined, are consistent with those used in the contemporary scientific literature, for example, the Journal of the American Chemical Society or the Journal of Biological Chemistry. Specifically, but without limitation, the following abbreviations may be used in the examples and throughout the specification:

g (grams); mg (milligrams);

L (liters); mL (milliliters);

μL (microliters); psi (pounds per square inch);

M (molar); mM (millimolar);

Hz (Hertz); MHz (megahertz);

mol (moles); mmol (millimoles);

RT (room temperature); h (hours);

min (minutes); TLC (thin layer chromatography);

mp (melting point); RP (reverse phase);

Tᵣ (retention time); TFA (trifluoroacetic acid);

TEA (triethylamine); THF (tetrahydrofuran);

TFAA (trifluoroacetic anhydride); CD₃OD (deuterated methanol);

CDCl₃ (deuterated chloroform); DMSO (dimethylsulfoxide);

SiO₂ (silica); atm (atmosphere);

EtOAc (ethyl acetate); CHCl₃ (chloroform);

HCl (hydrochloric acid); Ac (acetyl);

DMF (N,N-dimethylformamide); Me (methyl);

Cs₂CO₃ (cesium carbonate); EtOH (ethanol);

Et (ethyl); tBu (tert-butyl);

MeOH (methanol).

Unless otherwise indicated, all temperatures are expressed in ° C. (degrees Centigrade). All reactions conducted at room temperature unless otherwise noted.

¹H NMR spectra were recorded on a Varian VXR-300, a Varian Unity-300, a Varian Unity-400 instrument, or a General Electric QE-300. Chemical shifts are expressed in parts per million (ppm, δ units). Coupling constants are in units of hertz (Hz). Splitting patterns describe apparent multiplicities and are designated as s (singlet), d (doublet), t (triplet), q (quartet), m (multiplet), or br (broad).

Mass spectra were obtained on Micromass Platform or ZMD mass spectrometers from Micromass Ltd., Altricham, UK, using either Atmospheric Chemical Ionization (APCI) or Electrospray Ionization (ESI).

Analytical thin layer chromatography was used to verify the purity of intermediate(s) which could not be isolated or which were too unstable for full characterization as well as to follow the progress of reaction(s).

The absolute configuration of compounds were assigned by Ab Initio Vibrational Circular Dichroism (VCD) Spectroscopy^{1,2}. The experimental VCD spectrum were acquired in CDCl₃ using a Bomem ChirallR™ VCD spectrometer operating between 2000 and 800 cm⁻¹. The Gaussian 98 suite of computational programs were used to calculate model VCD spectrums³. The stereochemical assignment were made by comparing this experimental spectrum to the VCD spectrum calculated for a model structure with (R) or (S)-configuration.
1. J. R. Chesseman, et al, Chem. Phys. Lett. 252 (1996) 211.
2. P. J. Stephens and F. J. Devlin, Chirality 12 (2000) 172.
3. Gaussian 98, Revision A. 11.4, M. J. Frisch, et al, Gaussian, Inc., Pittsburgh Pa., 2002.

Compounds of formula (I) can be prepared by any suitable method known to one skilled in the art or by the process outlined below:

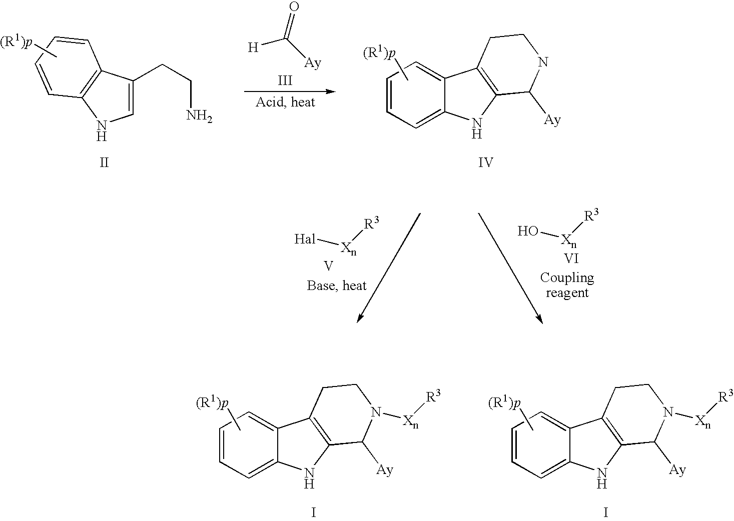

A compound of formula (IV) can be prepared by a Pictet-Spangler type reaction between a tryptamine of formula (II) and a benzaldehyde of formula (III). The reaction can be performed in a suitable solvent in the presence of an acid and optionally with heating. Suitable solvents include halogenated hydrocarbon solvents (e.g. dichloromethane), aromatic hydrocarbon solvents (e.g. toluene) and the like. Suitable acids include hydrochloric acid, trifluoroacetic acid. This reaction can also be run under conditions that facilitate removal of water, e.g. in toluene using a Dean Stark apparatus. Compounds of formula II and formula III are commercially available or can be prepared as described in known literature.

Compound of formula (I) can be prepared by condensation of compound of formula (IV) and compound of formula (V) in a suitable solvent optionally in the presence of base, optionally with heating. Suitable solvents include halogenate solvents (e.g. dichloromethane), aromatic hydrocarbon solvents (e.g. toluene), N,N-dimethylformamide, dimethylsulfoxide, acetonitrile, nitromethane and the like. Suitable bases include triethylamine, diisopropylethylamine, dimethylaminopyridine, pyridine and the like.

Alternatively, compounds of formula (I) can be prepared by reaction of compound of formula (IV) and compound of formula (VI). The reaction can be carried out in the presence of suitable copling reagents, such as 1-(3-dimethylaminopropyl)-3-ethylcarbodiimide hydrochloride and 1-hydroxybenzotriazole in a suitable solvent. Suitable solvents include dimethylformamide, dichloromethane and the like.

### EXAMPLES

### Example 1

### 6-Chloro-1-(4-methylphenyl)-2,3,4,9-tetrahydro-1H-β-carboline

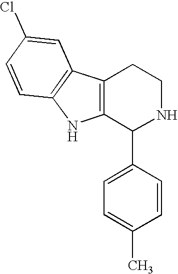

A suspension of 5-chlorotryptamine hydrochloride (3.70 g, 16.0 mmol) and p-tolualdehyde (1.80 mL, 15.2 mmol) in glacial acetic acid (50 mL) was heated at 80° C. overnight. The resulting precipitate was collected by filtration, rinsed with hexane and dried briefly. The solid was then stirred with dichloromethane and 10% aqueous sodium carbonate until all of the solids had dissolved. The layers were separated, and the aqueous layer was washed again with dichloromethane. The combined organic layers were washed with brine, dried over magnesium sulfate and concentrated to afford 6-chloro-1-(4-methylphenyl)-2,3,4,9-tetrahydro-1H-β-carboline (4.37 g, 92% yield) as a white solid. ¹H NMR (DMSO-d₆): δ 10.60 (s, 1H), 7.46 (d, 1H), 7.25-7.17 (m, 5H), 7.01 (dd, 1H), 5.06 (s, 1H), 3.09 (m, 1H), 2.94 (m, 1H), 2.71 (m, 3H), 2.32 (s, 3H); MS m/z 297 (M+1).

### Example 2

### Methyl 6-chloro-1-(4-methylphenyl)-1,3,4,9-tetrahydro-2H-β-carboline-2-carboxylate

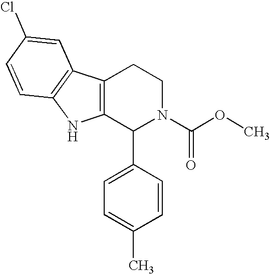

To a solution of 6-chloro-1-(4-methylphenyl)-2,3,4,9-tetrahydro-1H-β-carboline (87 mg, 0.29 mmol) and 4-(dimethylamino)pyridine (54 mg, 0.44 mmol) in dichloromethane (3 mL) was added methyl chloroformate (23 μL, 0.29 mmol). After stirring at room temperature for 3 h, the reaction was diluted with aqueous sodium bicarbonate and stirred for 15 min, then filtered through a hydrophobic frit. The aqueous layer was stirred with additional dichloromethane and the filtration was repeated. The combined organic layers were concentrated and purified by silica gel chromatography using a gradient of 0 to 40% ethyl acetate in hexane to afford methyl 6-chloro-1-(4-methylphenyl)-1,3,4,9-tetrahydro-2H-β-carboline-2-carboxylate (102 mg, 98%) as a white solid. ¹H NMR (DMSO-d₆): δ 11.12 (bs, 1H), 7.51 (d, 1H), 7.30 (d, 1H), 7.17 (m, 2H), 7.11-7.06 (m, 3H), 6.35 (br, 1H), 4.15 (br, 1H), 3.69 (s, 3H), 3.04 (m, 1H), 2.77 (m, 2H), 2.28 (s, 3H); MS m/z 355 (M+1).

### Example 3

### 6-Chloro-1-(4-methylphenyl)-2-(3-phenylpropanoyl)-2,3,4,9-tetrahydro-1H-β-carboline

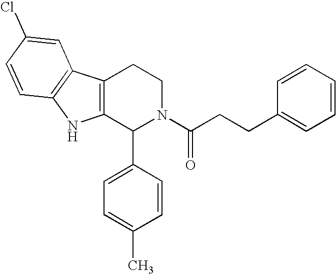

6-Chloro-1-(4-methylphenyl)-2-(3-phenylpropanoyl)-2,3,4,9-tetrahydro-1H-β-carboline was prepared from 6-chloro-1-(4-methylphenyl)-2,3,4,9-tetrahydro-1H-β-carboline (87 mg, 0.29 mmol) and hydrocinnamoyl chloride in a similar manner as described above to give a white solid (115 mg, 91%). ¹H NMR (DMSO-d₆): δ11.18 and 10.95 (s, 1H, major and minor conformers), 7.50 (d, 1H), 7.31-7.21 (m, 5H), 7.16-7.06 (m, 6H), 6.85 and 6.24 (s, 1H, major and minor conformers), 4.71 and 4.00 (m, 1H, minor and major conformers), 3.14 (m, 1H), 2.91-2.67 (m, 6H), 2.28 (s, 3H); MS m/z 429 (M+1).

### Example 4

### Phenylmethyl 6-chloro-1-(4-methylphenyl)-1,3,49-tetrahydro-2H-β-carboline-2-carboxylate

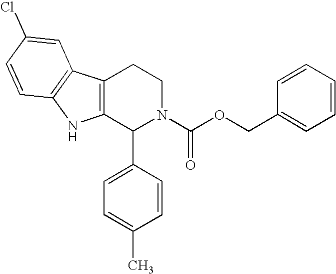

Phenylmethyl 6-chloro-1-(4-methylphenyl)-1,3,4,9-tetrahydro-2H-β-carboline-2-carboxylate was prepared from 6-chloro-1-(4-methylphenyl)-2,3,4,9-tetrahydro-1H-β-carboline (87 mg, 0.29 mmol) and benzyl chloroformate in a similar manner as described above to give an off-white solid (93 mg, 74%). ¹H NMR (DMSO-d₆): δ 11.10 (br, 1H), 7.52 (d, 1H), 7.39-7.06 (m, 11H), 6.35 (br, 1H), 5.22-5.12 (m, 2H), 4.21 (br, 1H), 3.07 (m, 1H), 2.79 (m, 2H), 2.28 (s, 3H); MS m/z 431 (M+1).

### Example 5

### 6-Fluoro-1-(4-methylphenyl)-2,3,4,9-tetrahydro-1H-β-carboline

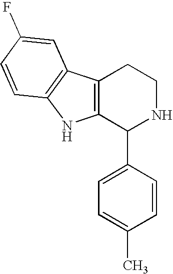

6-Fluoro-1-(4-methylphenyl)-2,3,4,9-tetrahydro-1H-β-carboline was prepared from 5-fluorotryptamine hydrochloride (0.52 g, 2.44 mmol) in a similar manner as described above to give a white solid (0.53 g, 78%). ¹H NMR (DMSO-d₆): δ 10.42 (s, 1H), 7.17-7.13 (m, 6H), 6.82-6.77 (m, 1H), 5.00 (s, 1H), 3.02 (m, 1H), 2.89 (m, 1H), 2.65 (m, 3H), 2.27 (s, 3H); MS m/z 281 (M+1).

### Example 6

### Methyl 6-fluoro-1-(4-methylphenyl)-1,3,4,9-tetrahydro-2H-β-carboline-2-carboxylate

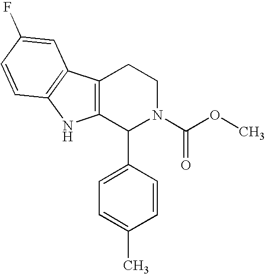

Methyl 6-fluoro-1-(4-methylphenyl)-1,3,4,9-tetrahydro-2H-β-carboline-2-carboxylate was prepared from 6-fluoro-1-(4-methylphenyl)-2,3,4,9-tetrahydro-1H-β-carboline (63 mg, 0.20 mmol) and methyl chloroformate in a similar manner as described above to give a white solid (48 mg, 63%). ¹H NMR (DMSO-d₆): δ 11.01 (bs, 1H), 7.29-7.06 (m, 6H), 6.91 (m, 1H), 6.35 (br, 1H), 4.15 (br, 1H), 3.69 (s, 3H), 3.04 (m, 1H), 2.75 (m, 2H), 2.28 (s, 3H); MS m/z 339 (M+1).

### Example 7

### 6-Fluoro-1-(4-methylphenyl)-2-(3-phenylpropanoyl)-2,3,4,9-tetrahydro-1H-β-carboline

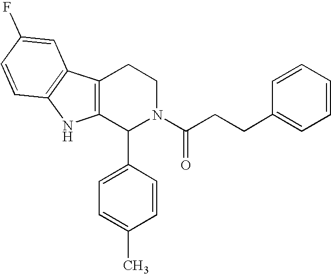

6-Fluoro-1-(4-methylphenyl)-2-(3-phenylpropanoyl)-2,3,4,9-tetrahydro-1H-β-carboline was prepared from 6-fluoro-1-(4-methylphenyl)-2,3,4,9-tetrahydro-1H-β-carboline (63 mg, 0.20 mmol) and hydrocinnamoyl chloride in a similar manner as described above to give a white foam (85 mg, 91%). ¹H NMR (DMSO-d₆): δ 11.07 and 10.84 (s, 1H, major and minor conformers), 7.30-7.03 (m, 11H), 6.91 (m, 1H), 6.84 and 6.23 (s, 1H, major and minor conformers), 4.71 and 4.03 (m, 1H, minor and major conformers), 3.14 (m, 1H), 2.89-2.67 (m, 6H), 2.28 (s, 3H); MS m/z 413 (M+1).

### Example 8

### Phenylmethyl 6-fluoro-1-(4-methylphenyl)-1,3,4,9-tetrahydro-2H-β-carboline-2-carboxylate

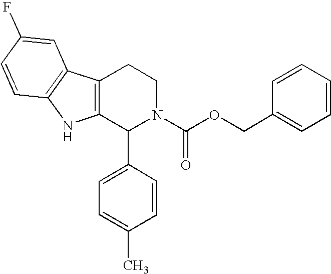

Phenylmethyl 6-fluoro-1-(4-methylphenyl)-1,3,4,9-tetrahydro-2H-β-carboline-2-carboxylate was prepared from 6-fluoro-1-(4-methylphenyl)-2,3,4,9-tetrahydro-1H-β-carboline (63 mg, 0.20 mmol) and benzyl chloroformate in a similar manner as described above to give a white foam (83 mg, 89%). ¹H NMR (DMSO-d₆): δ 10.98 (br, 1H), 7.39-7.10 (m, 11H), 6.91 (m, 1H), 6.34 (br, 1H), 5.22-5.10 (m, 2H), 4.21 (br, 1H), 3.07 (m, 1H), 2.76 (s, 2H), 2.28 (s, 3H); MS m/z 415 (M+1).

### Example 9

### 6-Bromo-1-(4-methylphenyl)-2,3,4,9-tetrahydro-1H-β-carboline

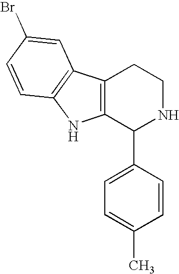

6-Bromo-1-(4-methylphenyl)-2,3,4,9-tetrahydro-1H-β-carboline was prepared from 5-bromotryptamine hydrochloride (0.47 g, 1.78 mmol) in a similar manner as described above to give a white solid (0.49 g, 80%). ¹H NMR (DMSO-d₆): δ 10.59 (s, 1H), 7.57 (d, 1H), 7.18-7.08 (m, 6H), 5.03 (s, 1H), 3.05 (m, 1H), 2.91 (m, 1H), 2.68 (m, 3H), 2.29 (s, 3H); MS m/z 341 (M+1).

### Example 10

### Methyl 6-bromo-1-(4-methylphenyl)-1,3,4,9-tetrahydro-2H-β-carboline-2-carboxylate

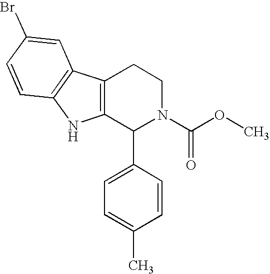

Methyl 6-bromo-1-(4-methylphenyl)-1,3,4,9-tetrahydro-2H-β-carboline-2-carboxylate was prepared from 6-bromo-1-(4-methylphenyl)-2,3,4,9-tetrahydro-1H-β-carboline (60 mg, 0.18 mmol) and methyl chloroformate in a similar manner as described above to give a white foam (49 mg, 70%). ¹H NMR (DMSO-d₆): δ 11.14 (s, 1H), 7.65 (d, 1H), 7.26 (m, 1H), 7.20-7.16 (m, 3H), 7.10 (m, 2H), 6.30 (br, 1H), 4.14 (br, 1H), 3.69 (s, 3H), 3.04 (m, 1H), 2.78 (m, 2H), 2.28 (s, 3H); MS m/z 399 (M+1).

### Example 11

### 6-Bromo-1-(4-methylphenyl)-2-(3-phenylpropanoyl)-2,3,4,9-tetrahydro-1H-β-carboline

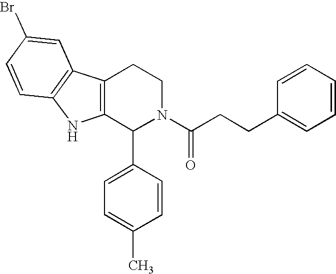

6-Bromo-1-(4-methylphenyl)-2-(3-phenylpropanoyl)-2,3,4,9-tetrahydro-1H-β-carboline was prepared from 6-bromo-1-(4-methylphenyl)-2,3,4,9-tetrahydro-1H-β-carboline (60 mg, 0.18 mmol) and hydrocinnamoyl chloride in a similar manner as described above to give a white solid (71 mg, 86%). ¹H NMR (DMSO-d₆): δ 11.19 and 10.96 (s, 1H, major and minor conformers), 7.64 (d, 1H), 7.29-7.10 (m, 9H), 7.06 (m, 2H), 6.84 and 6.24 (s, 1H, major and minor conformers), 4.70 and 4.03 (m, 1H, minor and major conformers), 3.14 (m, 1H), 2.89-2.68 (m, 6H), 2.28 (s, 3H); MS m/z 473 (M+1).

### Example 12

### Phenylmethyl 6-bromo-1-(4-methylphenyl)-1,3,4,9-tetrahydro-2H-β-carboline-2-carboxylate

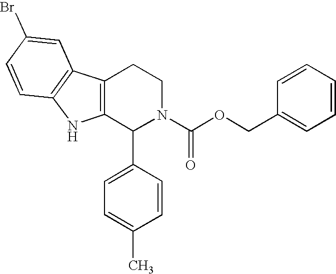

Phenylmethyl 6-bromo-1-(4-methylphenyl)-1,3,4,9-tetrahydro-2H-β-carboline-2-carboxylate was prepared from 6-bromo-1-(4-methylphenyl)-2,3,4,9-tetrahydro-1H-β-carboline (60 mg, 0.18 mmol) and benzyl chloroformate in a similar manner as described above to give a white foam (83 mg, 99%). ¹H NMR (DMSO-d₆): δ 11.11 (br, 1H), 7.66 (s, 1H), 7.48-7.09 (m, 11H), 6.35 (br, 1H), 5.22-5.12 (m, 2H), 4.21 (br, 1H), 3.07 (m, 1H), 2.78 (m, 2H), 2.28 (s, 3H); MS m/z 475 (M+1).

### Example 13

### (1R)-6-Chloro-1-(4-methylphenyl)-2-(3-phenylpropanoyl)-2,3,4,9-tetrahydro-1H-β-carboline

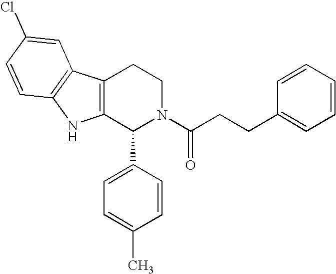

(1R)-6-Chloro-1-(4-methylphenyl)-2-(3-phenylpropanoyl)-2,3,4,9-tetrahydro-1H-β-carboline was prepared by separation of (1R/1S)-6-chloro-1-(4-methylphenyl)-2-(3-phenylpropanoyl)-2,3,4,9-tetrahydro-1H-β-carboline by supercritical fluid chromatography (Diacel OD-H, 30% methanol, 1500 psi, 40° C., 2 mL/min, retention time 10.0 min). Stereochemistry was assigned by vibrational circular dichroism.

### Example 14

### (1S)-6-Chloro-1-(4-methylphenyl)-2-(3-phenylpropanoyl)-2,3,4,9-tetrahydro-1H-β-carboline

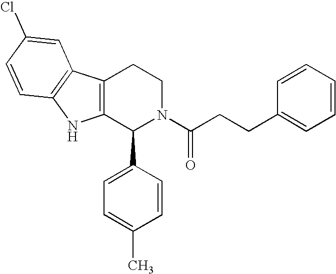

(1S)-6-Chloro-1-(4-methylphenyl)-2-(3-phenylpropanoyl)-2,3,4,9-tetrahydro-1H-β-carboline was prepared by separation of (1R/1S)-6-chloro-1-(4-methylphenyl)-2-(3-phenylpropanoyl)-2,3,4,9-tetrahydro-1H-β-carboline by supercritical fluid chromatography (Diacel OD-H, 30% methanol, 1500 psi, 40° C., 2 mL/min, retention time 13.0 min). Stereochemistry was assigned by vibrational circular dichroism.

### Example 15

### 1-(4-Methylphenyl)-2-[(phenylmethyl)sulfonyl]-2,3,4,9-tetrahydro-1H-β-carboline

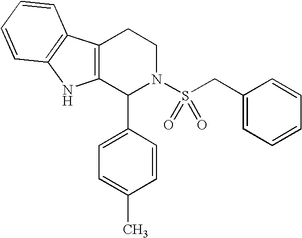

¹H NMR (DMSO-d₆): δ10.82 (s, 1H), 7.47 (d, 1H), 7.28 (m, 2H), 7.21-7.00 (m, 10H), 5.96 (s, 1H), 4.29 (s, 2H), 3.78 (dd, 1H), 3.13 (m, 1H), 2.87 (m, 1H), 2.75 (m, 1H), 2.29 (s, 3H); MS m/z 417 (M+1).

### Example 16

### 1-(4-Methylphenyl)-2-(methylsulfonyl)-2,3,4,9-tetrahydro-1H-β-carboline

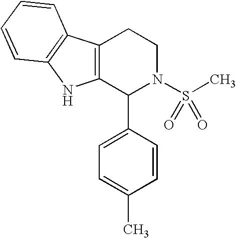

¹H NMR (DMSO-d₆): δ 10.89 (s, 1H), 7.48 (d, 1H), 7.29 (d, 1H), 7.20-6.98 (m, 6H), 6.02 (s, 1H), 3.87 (dd, 1H), 3.18 (m, 1H), 2.97 (m, 1H), 2.80 (m, 1H), 2.78 (s, 3H), 2.29 (s, 3H); MS m/z 341 (M+1).

### Example 17

### (4-{[1-(4-Methylphenyl)-1,3,4,9-tetrahydro-2H-b-carbolin-2-yl]sulfonyl}phenyl)amine

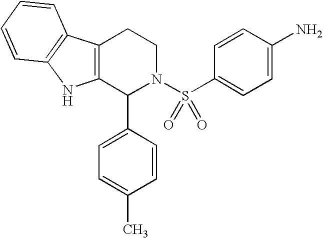

¹H NMR (DMSO-d₆): δ 10.78 (s, 1H), 7.34 (m, 3H), 7.24 (d, 1H), 7.14 (d, 2H), 7.03 (m, 3H), 6.93 (m, 1H), 6.43 (d, 2H), 6.09 (s, 1H), 5.88 (br, 2H), 3.80 (dd, 1H), 3.10 (m, 1H), 2.59-2.39 (m, 2H), 2.28 (s, 3H); MS m/z 418 (M+1).

### Example 18

### 2-Acetyl-1-(4-methylphenyl)-2,3,4,9-tetrahydro-1H-β-carboline

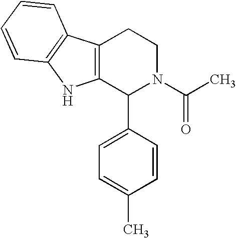

¹H NMR (DMSO-d₆): δ 10.94 and 10.79 (s, 1H, major and minor conformers), 7.46 (d, 1H), 7.28 (d, 1H), 7.20-6.97 (m, 6H), 6.80 and 6.14 (s, 1H, major and minor conformers), 4.68 and 3.94 (m, 1H, minor and major conformers), 3.21 (m, 1H), 2.91-2.72 (m, 2H), 2.27 (s, 3H), 2.13 (s, 3H); MS m/z 305 (M+1).

### Example 19

### 1-(4-Methylphenyl)-2-(3-phenylpropanoyl)-2,3,4,9-tetrahydro-1H-β-carboline

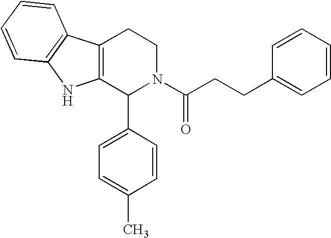

¹H NMR (CDCl₃): δ 7.88 (s, 1H), 7.52 (d, 1H), 7.29-7.09 (m, 11H), 7.02 and 5.92 (s, 1H, major and minor conformers), 4.95 and 3.87 (m, 1H, minor and major conformers), 3.36 (m, 1H), 3.00 (m, 2H), 2.83 (d, 2H), 2.71 (m, 2H), 2.33 (s, 3H); MS m/z 395 (M+1).

### Example 20

### 1-(4-Methylphenyl)-2-(3-phenyl-2-propynoyl)-2,3,4,9-tetrahydro-1H-b-carboline

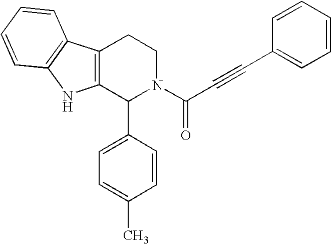

¹H NMR (CDCl₃): δ 7.75 (s, 1H), 7.63-7.55 (m, 2H), 7.46-7.13 (m, 10H), 6.96 and 6.78 (s, 1H, major and minor conformers), 4.83 and 4.62 (dd, 1H, minor and major conformers), 3.56 (m, 1H), 3.13-2.86 (m, 2H), 2.34 (s, 3H); MS m/z 391 (M+1).

### Example 21

### 1-(4-Methylphenyl)-2-[3-(3-pyridinyl)propanoyl]-2,3,4,9-tetrahydro-1H-β-carboline

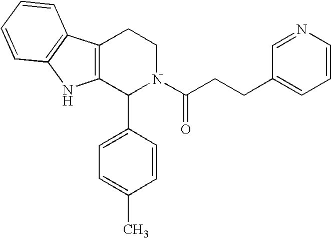

¹H NMR (DMSO-d₆): δ10.95 and 10.75 (s, 1H, major and minor conformers), 8.50 (s, 1H), 8.38 (d, 1H), 7.69 (d, 1H), 7.45 (d, 1H), 7.27 (m, 2H), 7.15 (m, 2H), 7.08 (m, 3H), 6.99 (t, 1H), 6.83 and 6.23 (s, 1H, major and minor conformers), 4.72 and 4.03 (m, 1H, minor and major conformers), 3.17 (m, 1H), 2.92-2.73 (m, 6H), 2.27 (s, 3H); MS m/z 396 (M+1).

### Example 22

### Phenylmethyl {2-[1-(4-methylphenyl)-1,3,4,9-tetrahydro-2H-b-carbolin-2-yl]-2-oxoethyl}carbamate

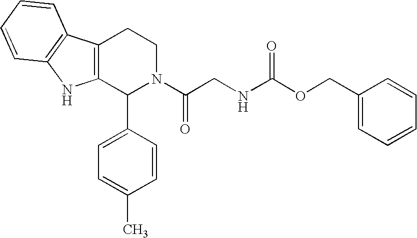

¹H NMR (DMSO-d₆): δ 10.96 and 10.81 (s, 1H, major and minor conformers), 7.47 (d, 1H), 7.42-7.28 (m, 6H), 7.16-6.99 (m, 6H), 6.75 and 6.21 (s, 1H, major and minor conformers), 5.04 (m, 2H), 4.01-3.96 (m, 3H), 3.21 (m, 1H), 2.85 (m, 2H), 2.27 (s, 3H); MS m/z 454 (M+1).

### Example 23

### 6-(Methyloxy)-1-(4-methylphenyl)-2-(3-phenylpropanoyl)-2,3,4,9-tetrahydro-1H-β-carboline

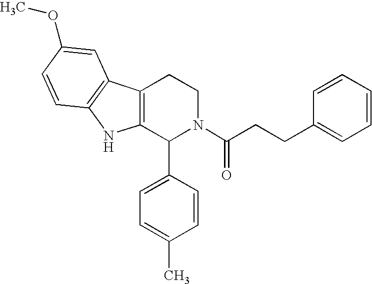

¹H NMR (DMSO-d₆): δ 10.77 and 10.56 (s, 1H, major and minor conformers), 7.26-7.21 (m, 4H), 7.18-7.13 (m, 4H), 7.07 (m, 2H), 6.94 (d, 1H), 6.82 and 6.20 (s, 1H, major and minor conformers), 6.71 (dd, 1H), 4.72 and 4.00 (m, 1H, minor and major conformers), 3.76 (s, 3H), 3.15 (m, 1H), 2.90-2.68 (m, 6H), 2.28 (s, 3H); MS m/z 425 (M+1).

### Example 24

### 8-Methyl-1-(4-methylphenyl)-2-(3-phenylpropanoyl)-2,3,4,9-tetrahydro-1H-β-carboline

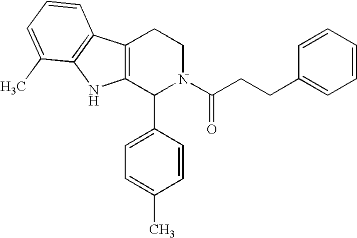

¹H NMR (DMSO-d₆): δ 10.86 and 10.73 (s, 1H, major and minor conformers), 7.28-7.22 (m, 5H), 7.18-7.07 (m, 5H), 6.93-6.86 (m, 3H), 6.17 (s, 1H, minor conformer), 4.60 and 3.99 (m, 1H, minor and major conformers), 3.14 (m, 1H), 2.90-2.67 (m, 6H), 2.39 (s, 3H), 2.28 (s, 3H); MS m/z 409 (M+1).

### Example 25

### 6-Methyl-1-(4-methylphenyl)-2-(3-phenylpropanoyl)-2,3,4,9-tetrahydro-1H-β-carboline

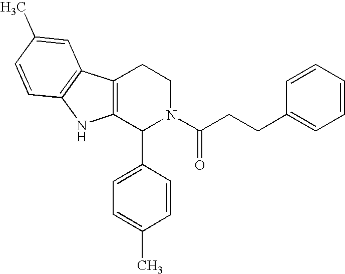

¹H NMR (DMSO-d₆): δ 10.80 and 10.60 (s, 1H, major and minor conformers), 7.25-7.12 (m, 9H), 7.07 (m, 2H), 6.89 (d, 1H), 6.82 and 6.20 (s, 1H, major and minor conformers), 4.71 and 4.00 (m, 1H, minor and major conformers), 3.14 (m, 1H), 2.90-2.67 (m, 6H), 2.37 (s, 3H), 2.27 (s, 3H); MS m/z 409 (M+1).

### Example 26

### 7-Fluoro-1-(4-methylphenyl)-2-(3-phenylpropanoyl)-2,3,4,9-tetrahydro-1H-β-carboline

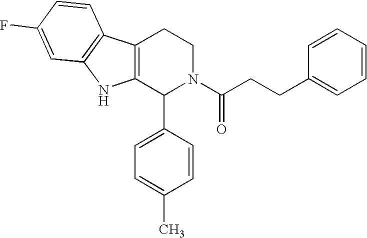

¹H NMR (DMSO-d₆): δ 11.07 and 10.83 (s, 1H, major and minor conformers), 7.43 (m, 1H), 7.26-7.04 (m, 10H), 6.86 (m, 1H), 6.82 and 6.21 (s, 1H, major and minor conformers), 4.73 and 4.00 (m, 1H, minor and major conformers), 3.14 (m, 1H), 2.89-2.68 (m, 6H), 2.28 (s, 3H); MS m/z 413 (M+1).

### Example 27

### 7-(Methyloxy)-1-(4-methylphenyl)-2-(3-phenylpropanoyl)-2,3,4,9-tetrahydro-1H-β-carboline

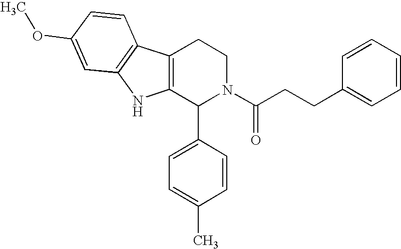

¹H NMR (DMSO-d₆): δ 10.77 and 10.55 (s, 1H, major and minor conformers), 7.31 (d, 1H), 7.24 (m, 4H), 7.19-7.07 (m, 5H), 6.80 (s, 2H), 6.65 (dd, 1H), 6.17 (s, 1H, minor conformer), 4.69 and 3.98 (m, 1H, minor and major conformers), 3.75 (s, 3H), 3.12 (m, 1H), 2.89-2.67 (m, 6H), 2.28 (s, 3H); MS m/z 425 (M+1).

### Example 28

### (1R/1S)-1-(2,3-dihydro-1-benzofuran-5-yl)-2,3,4,9-tetrahydro-1H-β-carboline

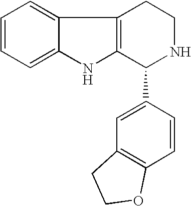

A solution of tryptamine (0.325 g, 2.03 mmol) and 2,3-dihydro-1-benzofuran-5-carbaldehyde (0.250 g, 1.69 mmol) in glacial acetic acid (10 mL) was heated to 100° C. with stirring. After 2.5 h the solution was treated with an additional portion of 2,3-dihydro-1-benzofuran-5-carbaldehyde (0.062 g, 0.418 mmol) and stirred at 0° C. for an additional 3 h. The dark brown solution was cooled to RT and stirred overnight. The solvent was removed by concentration at reduced pressure and the residue dissolved in dichloromethane. The solution was washed twice with 10% aqueous sodium bicarbonate followed by aqueous brine. The solution was then treated with magnesium sulfate and a small portion of activated charcoal and heated to boiling for 5 minutes. After cooling to RT, the mixture was filtered through celite and the filtrate evaporated to dryness to afford 0.590 g of a yellow-brown foam. The crude product was purified by flash chromatography (silica gel, 95:5 dichloromethane/2M ammonia in methanol) to give (1R/1S)-1-(2,3-dihydro-1-benzofuran-5-yl)-2,3,4,9-tetrahydro-1H-β-carboline (0.460 g, 94%) as a yellow powder. ¹H NMR (DMSO-d₆): δ 10.32 (s, 1H), 7.36 (d, 1H), 7.20 (d, 1H), 7.09 (s, 1H), 7.01-6.88 (m, 3H), 6.69 (d, 1H), 4.99 (s, 1H), 4.49 (t, 2H), 3.17-3.00 (m, 3H), 2.90 (m, 1H), 2.79-2.58 (m, 3H). MS m/z 291 (M+1).

### Example 29

### (1R/1S)-1-(2,3-dihydro-1-benzofuran-5-yl)-2-[(2E)-3-phenyl-2-propenoyl]-2,3,4,9-tetrahydro-1H-β-carboline

### Example 30

### (1R)-1-(2,3-dihydro-1-benzofuran-5-yl)-2-[(2E)-3-phenyl-2-propenoyl]-2,3,4,9-tetrahydro-1H-β-carboline

### Example 31

### (1S)-1-(2,3-dihydro-1-benzofuran-5-yl)-2-[(2E)-3-Phenyl-2-propenoyl]-2,3,4,9-tetrahydro-1H-β-carboline

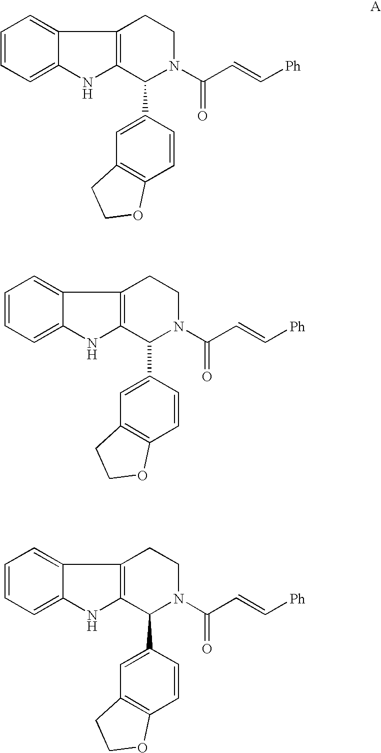

solution of (1R/1S)-1-(2,3-dihydro-1-benzofuran-5-yl)-2,3,4,9-tetrahydro-1H-β-carboline (0.250 g, 0.861 mmol) and N,N-diisopropylethylamine (0.300 mL, 1.72 mmol) in 10 mL of anhydrous dichloromethane was treated with cinnamoyl chloride (0.159 g, 0.947 mmol) and the resulting solution was stirred at RT. After 2.5 h, the solution was diluted with dichloromethane, washed with 5% aqueous citric acid (2×), 10% aqueous sodium bicarbonate (2×), dried over magnesium sulfate and concentrated to dryness at reduced pressure. The crude product was subjected to flash chromatography (silica gel, 97:3 dichloromethane/methanol) to afford 0.330 g (91%) (1R/1S)-1-(2,3-dihydro-1-benzofuran-5-yl)-2-[(2E)-3-phenyl-2-propenoyl]-2,3,4,9-tetrahydro-1H-β-carboline (compound 2) as a light yellow foam. ¹H NMR (DMSO-d₆, 95° C.): δ 10.63 (br s, 1H), 7.69 (d, 2H), 7.54 (d, 1H), 7.49-7.22 (m, 5H), 7.14 (s, 1H), 7.08 (t, 1H), 7.03-6.97 (m, 2H), 6.84 (br s, 1H), 6.70 (d, 1H), 5.67 (s, 1H), 4.49 (t, 2H), 4.43 (m, 1H), 3.32 (m, 1H), 3.13 (t, 2H), 2.87 (m, 2H). MS m/z 421 (M+1). A 223 mg portion of the racemic product was subjected to supercritical fluid chiral chromatography (Chiralpak AS-H 5 μM column, 3:1 MeOH/CHCl₃ mobile phase, 40° C., 180 bars, flow rate=180 g/minute) to afford 105 mg of (1R)-1-(2,3-dihydro-1-benzofuran-5-yl)-2-[(2E)-3-phenyl-2-propenoyl]-2,3,4,9-tetrahydro-1H-β-carboline (Example 30) eluting at 1.14 minutes and 99 mg of (1S)-1-(2,3-dihydro-1-benzofuran-5-yl)-2-[(2E)-3-phenyl-2-propenoyl]-2,3,4,9-tetrahydro-1H-β-carboline (Example 31) eluting at 1.88 minutes. Both compounds were determined to be enantiomerically pure by analytical chiral SFC (ChiralCel OJ column, 30% MeOH mobile phase, 3000 psi, 40° C., flow rate=3 mL/minute). The retention times were 11.7 and 20.2 minutes for compounds 30 and 31 respectively. The absolute configurations were assigned using VCD spectroscopy.

Additionally, the following compounds may be prepared as described in Bioorg. Med. Chem. Lett, 2003, 13, 761-765 and WO 02/064590 A2:

| Example | Compound Name | Structure |
|---|---|---|
| 32 | 1-(1,3-benzodioxol-5-yl)-2-{4-[4-(methyloxy)phenyl]-2-pyrimidinyl}-2,3,4,9-tetrahydro-1H-β-carboline | 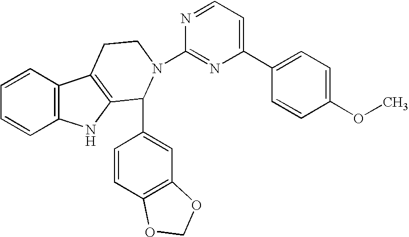 |
| 33 | 1-(1,3-benzodioxol-5-yl)-2-(2-pyrimidinyl)-2,3,4,9-tetrahydro-1H-β-carboline | 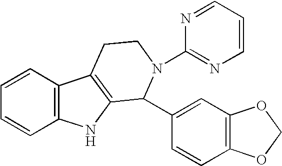 |
| 34 | 1-(1,3-benzodioxol-5-yl)-2-(4-phenyl-2-pyrimidinyl)-2,3,4,9-tetrahydro-1H-β-carboline | 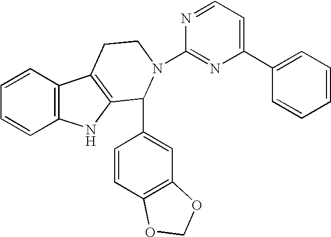 |
| 35 | 1-phenyl-2-(4-phenyl-2-pyrimidinyl)-2,3,4,9-tetrahydro-1H-β-carboline | 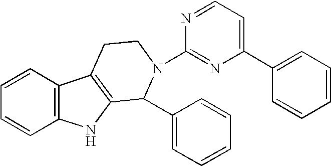 |
| 36 | 1-(2,3-dihydro-1-benzofuran-5-yl)-2-(4-phenyl-2-pyrimidinyl)-2,3,4,9-tetrahydro-1H-β-carboline | 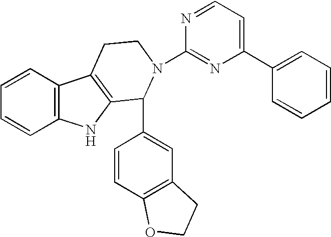 |
| 37 | 1-(2,3-dihydro-1-benzofuran-5-yl)-2-[4-(3-pyridinyl)-2-pyrimidinyl]-2,3,4,9-tetrahydro-1H-β-carboline | 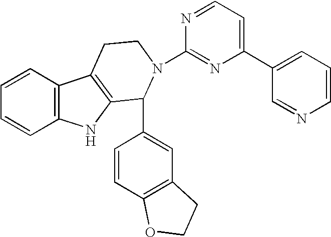 |
| 38 | 1-(2,3-dihydro-1-benzofuran-5-yl)-2-[4-(1H-imidazol-1-yl)-2-pyrimidinyl]-2,3,4,9-tetrahydro-1H-β-carboline | 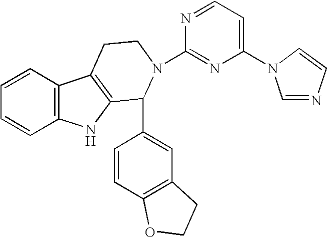 |
| 39 | 1-(2,3-dihydro-1-benzofuran-5-yl)-2-[4-(4-methyl-1H-imidazol-1-yl)-2-pyrimidinyl]-2,3,4,9-tetrahydro-1H-β-carboline | 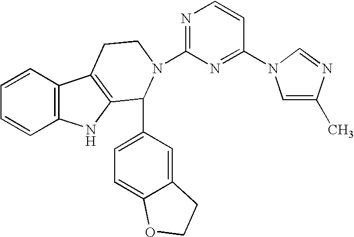 |
| 40 | 1-(2,3-dihydro-1-benzofuran-5-yl)-2-[4-(4-methyl-1H-imidazol-1-yl)-2-pyrimidinyl]-2,3,4,9-tetrahydro-1H-β-carboline | 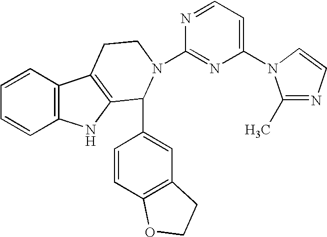 |

### Biological Experimentals and Data

Compounds of the current invention are believed useful in the treatment and/or prophylaxis of conditions and diseases associated with HPV infection. Activity mediated through HPV was determined using the following W-12 cellular assay.

### Cell Culture and Medium

The W12 cell line used contains HPV16 DNA and was derived from a low-grade cervical dysplasia tissue by Margaret Stanley and subsequently clonally selected by Paul Lambert (University of Wisconsin). One of these clones, W12-20850, contains 1000 copies of episomal HPV16 DNA and was used in the cell-based assay. W12-20850 cells were routinely cultured with a gamma-irradiated (6000 rads) feeder layer of 3T3 cells. Assays, however, were run in the absence of a 3T3 feeder layer. W12-20850 and 3T3 cells were routinely split when they were sub-confluent. W12-20850 were grown in W12 Medium which is constituted of 25% DMEM (Gibco BRL, Cat # 12430-047), 75% F12 Media (Gibco BRL, Cat # 11765-021) and 2.5% FBS. The additives include 24.0 mg/ml Adenine (Sigma, Cat # A-9795), 0.4 mg/ml Hydrocortisone (Calbiochem, Cat # 386698), 5.0 mg/ml Bovine Insulin (Sigma, Cat # 1-1882), 8.4 ng/ml cholera toxin (Fluka, Cat # 26694) and 10 ng/ml EGF (Invitrogen, Cat # 13247-051). 3T3 cells were grown in DMEM containing 10% FBS. Cell lines were incubated at 37° C., in the presence of 5% CO₂.

### Cell Based Assay

For the assay, W12-20850 cells were seeded into a 96 well plate-containing compound. Plates were incubated at 37° C. in the presence of 5% CO₂, for four days. On the fourth day, cells were lysed and the amount of episomal HPV-16 DNA was quantified using a non-radioactive hybrid capture technique with HPV-16 specific capture and detection probes. The percent inhibition relative to untreated control cells was then determined.

### Hybrid Capture

The hybrid capture assay is run in a 96 well plate format. Hybridization plates (Nunc Maxisorb Cat # 450320) were coated with a mixture of capture probe and ReactiBind solution for at least 4 hours and then washed with 0.2×SSC, 0.05% Tween-20 (SSCT) prior to blocking with 150 μl/well of 0.2 N NaOH, 1% Igepal, 10 mg/ml hsDNA for 6-8 hours. The hybridization was carried out by mixing 27 μl of lysed cells with 45 μl of denatured detection probe in 6M guanidine isothiocyanate. To prevent evaporation, 50 μl of mineral oil was added to each well. The plate was then heated to 90° C. for 6.5 minutes and the hybridization continued at 42° C. overnight. Assay plates were washed 6 times with SSC/T. Anti-digoxigenin HRP-conjugated Ab (Boehringer Mannheim 1207733, 1:5000) was incubated in the wells for 30 min at room temperature and washed with PBS/0.05% Tween-20. SuperSignal LBA substrate (Pierce Cat # 37070) was added, and chemiluminescence was measured using Wallac 1420 Victor plate reader.

| Example no | W12 activity | |
|---|---|---|
| 2 | 68 | nM |
| 3 | 60 | nM |
| 4 | 22 | nM |
| 6 | 365 | nM |
| 7 | 216 | nM |
| 8 | 446 | nM |
| 10 | 11 | nM |
| 11 | 20 | nM |
| 12 | 14 | nM |
| 13 | 8000 | nM |
| 14 | 15 | nM |
| 15 | 7 | uM |
| 16 | 24 | uM |
| 17 | 10 | uM |
| 18 | 5 | uM |
| 19 | 1 | uM |
| 20 | 26 | uM |
| 21 | 18 | uM |
| 22 | 4 | uM |
| 23 | 600 | nM |
| 24 | 2 | uM |
| 25 | 400 | nM |
| 26 | 2 | uM |
| 27 | 6 | uM |
| 29 | 3 | uM |
| 30 | >20 | uM |
| 31 | 2 | uM |
| 32 | 2 | uM |
| 33 | 5 | uM |
| 34 | 2 | uM |
| 35 | 2 | uM |
| 36 | 3 | uM |
| 37 | 4 | uM |
| 38 | 3 | uM |
| 39 | 2 | uM |
| 40 | 1 | uM |

Test compounds were employed in free or salt form.

All research complied with the principles of laboratory animal care (NIH publication No. 85-23, revised 1985) and GlaxoSmithKline policy on animal use.

Although specific embodiments of the present invention are herein illustrated and described in detail, the invention is not limited thereto. The above detailed descriptions are provided as exemplary of the present invention and should not be construed as constituting any limitation of the invention. Modifications will be obvious to those skilled in the art, and all modifications that do not depart from the spirit of the invention are intended to be included with the scope of the appended claims.

What is claimed is:

1. A method for the treatment or prophylaxis of conditions or disorders due to HPV infection comprising the administration of a compound of formula (I): 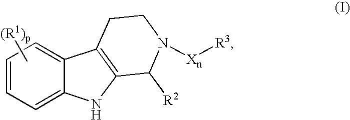
wherein:
each R¹ independently is halogen, haloalkyl, alkyl, alkenyl, alkynyl, cycloalkyl, cycloalkenyl, —R¹⁰cycloalkyl, Ay, AyR⁴, AyOR⁴—NHR¹⁰Ay, Het, HetR⁴, HetOR⁴, —NHHet, —NHR¹⁰Het, —OR⁴—OAy, —OHet, —R¹⁰OR⁴, —NR⁴R⁵, —NR⁴Ay, —R¹⁰NR⁴R⁵, —R¹⁰NR⁴Ay, —R¹⁰C(O)R⁴, —C(O)R⁴, —CO₂R⁴, —R¹⁰CO₂R⁴, —C(O)NR⁴R⁵, —C(O)Ay, —C(O)NR⁴Ay, —C(O)Het, —C(O)NHR¹⁰Het, —R¹⁰C(O)NR⁴R⁵, —C(S)NR⁴R⁵, —R¹⁰C(S)NR⁴R⁵, —R¹⁰NHC(NH)NR⁴R⁵, —C(NH)NR⁴R⁵, —R¹⁰C(NH)NR⁴R⁵, —S(O)₂NR⁴R⁵, —S(O)₂NR⁴Ay, —R¹⁰SO₂NHCOR⁴, —R¹⁰SO₂NR⁴R⁵, —R¹⁰SO₂R⁴, —S(O)ₘR⁴, cyano, nitro, or azido;
p is 0, 1, 2, 3, or 4;
n is 0 or 1;
m is 0, 1, or 2;
X is selected from a group consisting of C(O), C(O)O, C(O)Y, S(O), SO₂, S(O)Y, SO₂Y, C(O)OY, C(O)NHY, C(O)YN(H)C(O)OY;
each Y independently is optionally substituted alkylene, optionally substituted cycloalkylene, optionally substituted alkenylene, optionally substituted cycloalkenylene, or optionally substituted alkynylene;
R² is -Ay or -Het, each optionally substituted with one or more halogen, haloalkyl, alkyl, alkenyl, alkynyl, cycloalkyl, cycloalkenyl, —R¹⁰cycloalkyl, Ay, AyR⁴, AyOR⁴—NHR¹⁰Ay, Het, HetR⁴, HetOR⁴, —NHHet, —NHR¹⁰Het, —OR⁴, —OAy, —OHet, —R¹⁰OR⁴, —NR⁴R⁵, —NR⁴Ay, —R¹⁰NR⁴R⁵, —R¹⁰NR⁴Ay, —R¹⁰C(O)R⁴, —C(O)R⁴, —CO₂R⁴, —R¹⁰CO₂R⁴, —C(O)NR⁴R⁵, —C(O)Ay, —C(O)NR⁴Ay, —C(O)Het, —C(O)NHR¹⁰Het, —R¹⁰C(O)NR⁴R⁵, —C(S)NR⁴R⁵, —R¹⁰C(S)NR⁴R⁵, —R¹⁰NHC(NH)NR⁴R⁵, —C(NH)NR⁴R⁵, —R¹⁰C(NH)NR⁴R⁵, —S(O)₂NR⁴R⁵, —S(O)₂NR⁴Ay, —R¹⁰SO₂NHCOR⁴, —R¹⁰SO₂NR⁴R⁵, —R¹⁰SO₂R⁴, —S(O)ₘR⁴ cyano, nitro, or azido;
R³ is alkyl, -Ay or -Het, where Ay or Het may each be optionally substituted with one or more halogen, haloalkyl, alkyl, alkenyl, alkynyl, cycloalkyl, cycloalkenyl, —R¹⁰cycloalkyl, Ay, AyR⁴, AyOR⁴, —NHR¹⁰Ay, Het, HetR⁴, HetOR⁴, —NHHet, —NHR¹⁰Het, —OR⁴, —OAy, —OHet, —R¹⁰OR⁴, —NR⁴R⁵, —NR⁴Ay, —R¹⁰NR⁴R⁵, —R¹⁰NR⁴Ay, —R¹⁰C(O)R⁴, —C(O)R⁴, —CO₂R⁴, —R¹⁰CO₂R⁴, —C(O)NR⁴R⁵, —C(O)Ay, —C(O)NR⁴Ay, —C(O)Het, —C(O)NHR¹⁰Het, —R¹⁰C(O)NR⁴R⁵, —C(S)NR⁴R⁵, —R¹⁰C(S)NR⁴R⁵, —R¹⁰NHC(NH)NR⁴R⁵, —C(NH)NR⁴R⁵, —R¹⁰C(NH)NR⁴R⁵, —S(O)NR⁴R⁵, —S(O)₂NR⁴Ay, —R¹⁰SO₂NHCOR⁴, —R¹⁰SO₂NR⁴R⁵, —R¹⁰SO₂R⁴, —S(O)ₘR⁴, cyano, nitro, or azido;
each of R⁴ and R⁵ is independently selected from H or alkyl;
each R¹⁰ is an optionally substituted alkylene;
Ay represents an aryl group;
Het represents a 5- or 6-membered heterocyclyl or heteroaryl group; or pharmaceutically acceptable salts or solvates thereof.

2. The method of claim 1 wherein R¹ is selected from halogen, alkyl, cyano, nitro, —OR⁴, Het, —NR⁴R⁵, or —CONR⁴R⁵.

3. The method of claim 2 wherein R¹ is halogen or alkyl.

4. The method of claim 3 wherein R¹ is F, Cl, Br, or I.

5. The method of claim 4 wherein R¹ is Cl or Br.

6. The method of claim 1 wherein p is 1 and R¹ is substituted para to the depicted indole nitrogen atom.

7. The method of claim 1 wherein R² is Ay.

8. The method of claim 7 wherein Ay is phenyl substituted with one or two substituents.

9. The method of claim 8 wherein Ay is phenyl optionally substituted with halogen, alkyl, cyano, nitro, —OR⁴, Het, —NR⁴R⁵, or —CONR⁴R⁵.

10. The method of claim 9 wherein Ay is phenyl optionally substituted with halogen, alkyl, cyano, —OR⁴, —NR⁴R⁵, or —CONR⁴R⁵.

11. The method of claim 1 wherein R² is Het.

12. The method of claim 11 wherein Het is dihydrobenzofuran or piperonyl.

13. The method of claim 1 wherein n is 1 and X is selected from C(O), C(O)O, C(O)Y, C(O)OY, C(O)NHY, or SO₂Y.

14. The method of claim 13 wherein X is selected from C(O)Y, C(O)O, C(O)OY, or C(O)NHY.

15. The method of claim 13 wherein R³ is alkyl, Ay, or Het.

16. The method of claim 15 wherein R³ is alkyl or Ay.

17. The method of claim 1 wherein n is 0 and R³ is Het.

18. The method of claim 17 wherein R³ is Het optionally substituted with one or more of halogen, alkyl, cyano, nitro, —OR⁴, Het, Ay, —NR⁴R⁵, or —CONR⁴R⁵.

19. A method for the treatment or prophylaxis of conditions or disorders due to HPV infection comprising the administration of a compound selected from the group consisting of:
Methyl 6-chloro-1-(4-methylphenyl)-1,3,4,9-tetrahydro-2H-β-carboline-2-carboxylate;
6-Chloro-1-(4-methylphenyl)-2-(3-phenylpropanoyl)-2,3,4,9-tetrahydro-1H-β-carboline;
Phenylmethyl 6-chloro-1-(4-methylphenyl)-1,3,4,9-tetrahydro-2H-β-carboline-2-carboxylate;
Methyl 6-fluoro-1-(4-methylphenyl)-1,3,4,9-tetrahydro-2H-β-carboline-2-carboxylate;
6-Fluoro-1-(4-methylphenyl)-2-(3-phenylpropanoyl)-2,3,4,9-tetrahydro-1H-β-carboline;
Phenylmethyl 6-fluoro-1-(4-methylphenyl)-1,3,4,9-tetrahydro-2H-β-carboline-2-carboxylate;
Methyl 6-bromo-1-(4-methylphenyl)-1,3,4,9-tetrahydro-2H-β-carboline-2-carboxylate;
6-Bromo-1-(4-methylphenyl)-2-(3-phenylpropanoyl)-2,3,4,9-tetrahydro-1H-β-carboline;
Phenylmethyl 6-bromo-1-(4-methylphenyl)-1,3,4,9-tetrahydro-2H-β-carboline-2-carboxylate;
(1R)-6-Chloro-1-(4-methylphenyl)-2-(3-phenylpropanoyl)-2,3,4,9-tetrahydro-1H-β-carboline;
(1S)-6-Chloro-1-(4-methylphenyl)-2-(3-phenylpropanoyl)-2,3,4,9-tetrahydro-1H-β-carboline;
1-(4-Methylphenyl)-2-[(phenylmethyl)sulfonyl]-2,3,4,9-tetrahydro-1H-β-carboline;
1-(4-Methylphenyl)-2-(methylsulfonyl)-2,3,4,9-tetrahydro-1H-β-carboline;
(4-{[1-(4-Methylphenyl)-1,3,4,9-tetrahydro-2H-b-carbolin-2-yl]sulfonyl}phenyl)amine.
2-Acetyl-1-(4-methylphenyl)-2,3,4,9-tetrahydro-1H-β-carboline;
1-(4-Methylphenyl)-2-(3-phenylpropanoyl)-2,3,4,9-tetrahydro-1H-β-carboline;
1-(4-Methylphenyl)-2-(3-phenyl-2-propynoyl)-2,3,4,9-tetrahydro-1H-β-carboline;
1-(4-Methylphenyl)-2-[3-(3-pyridinyl)propanoyl]-2,3,4,9-tetrahydro-1H-β-carboline;
Phenylmethyl {2-[1-(4-methylphenyl)-1,3,4,9-tetrahydro-2H-β-carbolin-2-yl]-2-oxoethyl}carbamate;
6-(Methyloxy)-1-(4-methylphenyl)-2-(3-phenylpropanoyl)-2,3,4,9-tetrahydro-1H-β-carboline;
8-Methyl-1-(4-methylphenyl)-2-(3-phenylpropanoyl)-2,3,4,9-tetrahydro-1H-β-carboline;
6-Methyl-1-(4-methylphenyl)-2-(3-phenylpropanoyl)-2,3,4,9-tetrahydro-1H-β-carboline;
7-Fluoro-1-(4-methylphenyl)-2-(3-phenylpropanoyl)-2,3,4,9-tetrahydro-1H-βcarboline;
7-(Methyloxy)-1-(4-methylphenyl)-2-(3-phenylpropanoyl)-2,3,4,9-tetrahydro-1H-β-carboline;
(1R/1S)-1-(2,3-dihydro-1-benzofuran-5-yl)-2-[(2E)-3-phenyl-2-propenoyl]-2,3,4,9-tetrahydro-1H-β-carboline;
(1R)-1-(2,3-dihydro-1-benzofuran-5-yl)-2-[(2E)-3-phenyl-2-propenoyl]-2,3,4,9-tetrahydro-1H-β-carboline;
(1S)-1-(2,3-dihydro-1-benzofuran-5-yl)-2-[(2E)-3-phenyl-2-propenoyl]-2,3,4,9-tetrahydro-1H-β-carboline;
1-(1,3-benzodioxol-5-yl)-2-{4-[4-(methyloxy)phenyl]-2-pyrimidinyl}-2,3,4,9-tetrahydro-1H-β-carboline,
1-(1,3-benzodioxol-5-yl)-2-(2-pyrimidinyl)-2,3,4,9-tetrahydro-1H-β-carboline;
1-(1,3-benzodioxol-5-yl)-2-(4-phenyl-2-pyrimidinyl)-2,3,4,9-tetrahydro-1H-β-carboline;
1-phenyl-2-(4-phenyl-2-pyrimidinyl)-2,3,4,9-tetrahydro-1H-β-carboline;
1-(2,3-dihydro-1-benzofuran-5-yl)-2-(4-phenyl-2-pyrimidinyl)-2,3,4,9-tetrahydro-1H-β-carboline;
1-(2,3-dihydro-1-benzofuran-5-yl)-2-[4-(3-pyridinyl)-2-pyrimidinyl]-2,3,4,9-tetrahydro-1H-β-carboline;
1-(2,3-dihydro-1-benzofuran-5-yl)-2-[4-(1H-imidazol-1-yl)-2-pyrimidinyl]-2,3,4,9-tetrahydro-1H-β-carboline;
1-(2,3-dihydro-1-benzofuran-5-yl)-2-[4-(4-methyl-1H-imidazol-1-yl)-2-pyrimidinyl]-2,3,4,9-tetrahydro-1H-β-carboline;
1-(2,3-dihydro-1-benzofuran-5-yl)-2-[4-(4-methyl-1H-imidazol-1-yl)-2-pyrimidinyl]-2,3,4,9-tetrahydro-1H-β-carboline; and pharmaceutically acceptable salts or solvates thereof.

20. A method for the treatment or prophylaxis of conditions or disorders due to HPV infection comprising the administration of a compound selected from the group consisting of:
Methyl 6-chloro-1-(4-methylphenyl)-1,3,4,9-tetrahydro-2H-β-carboline-2-carboxylate;
6-Chloro-1-(4-methylphenyl)-2-(3-phenylpropanoyl)-2,3,4,9-tetrahydro-1H-β-carboline;
Phenylmethyl 6-chloro-1-(4-methylphenyl)-1,3,4,9-tetrahydro-2H-β-carboline-2-carboxylate;
Methyl 6-fluoro-1-(4-methylphenyl)-1,3,4,9-tetrahydro-2H-β-carboline-2-carboxylate;
6-Fluoro-1-(4-methylphenyl)-2-(3-phenylpropanoyl)-2,3,4,9-tetrahydro-1H-β-carboline;
Phenylmethyl 6-fluoro-1-(4-methylphenyl)-1,3,4,9-tetrahydro-2H-β-carboline-2-carboxylate;
Methyl 6-bromo-1-(4-methylphenyl)-1,3,4,9-tetrahydro-2H-β-carboline-2-carboxylate;
6-Bromo-1-(4-methylphenyl)-2-(3-phenylpropanoyl)-2,3,4,9-tetrahydro-1H-β-carboline;
Phenylmethyl 6-bromo-1-(4-methylphenyl)-1,3,4,9-tetrahydro-2H-β-carboline-2-carboxylate;
(1S)-6-Chloro-1-(4-methylphenyl)-2-(3-phenylpropanoyl)-2,3,4,9-tetrahydro-1H-β-carboline;
6-(Methyloxy)-1-(4-methylphenyl)-2-(3-phenylpropanoyl)-2,3,4,9-tetrahydro-1H-β-carboline; and
6-Methyl-1-(4-methylphenyl)-2-(3-phenylpropanoyl)-2,3,4,9-tetrahydro-1H-β-carboline; and pharmaceutically acceptable salts or solvates thereof.

21. A method for the treatment or prophylaxis of conditions or disorders due to HPV infection comprising the administration of a compound selected from the group consisting of:
Methyl 6-chloro-1-(4-methylphenyl)-1,3,4,9-tetrahydro-2H-β-carboline-2-carboxylate;
6-Chloro-1-(4-methylphenyl)-2-(3-phenylpropanoyl)-2,3,4,9-tetrahydro-1H-β-carboline;
Phenylmethyl 6-chloro-1-(4-methylphenyl)-1,3,4,9-tetrahydro-2H-β-carboline-2-carboxylate;
Methyl 6-bromo-1-(4-methylphenyl)-1,3,4,9-tetrahydro-2H-β-carboline-2-carboxylate;
6-Bromo-1-(4-methylphenyl)-2-(3-phenylpropanoyl)-2,3,4,9-tetrahydro-1H-β-carboline,
Phenylmethyl 6-bromo-1-(4-methylphenyl)-1,3,4,9-tetrahydro-2H-β-carboline-2-carboxylate; and
(1S)-6-Chloro-1-(4-methylphenyl)-2-(3-phenylpropanoyl)-2,3,4,9-tetrahydro-1H-β-carboline; and pharmaceutically acceptable salts or solvates thereof.

22. The method of claim 1 for the treatment or prophylaxis of diseases and conditions caused by oncogenic viruses, including adenoviruses, retroviruses, and viruses from the papovavirus family, including polyoma viruses and papilloma viruses.

23. The method of claim 22 wherein the condition or disease is warts, genital warts, cervical dysplasia, recurrent respiratory papillomatosis, or cancers associated with papillomavirus infection.

24. The method of claim 23 wherein the cancer is anogenital cancers, head and neck cancers, and skin cancers.

25. The method of claim 24 wherein
the anogenital cancers are cervical, anal and perianal, vulvar, vaginal, and penile cancers;
the head and neck cancers are oral pharyngeal region and esophagus cancers; and
the skin cancers are basal cell carcinoma and squamous cell carcinoma.

26. Use of a compound according to claim 1 in the manufacture of a medicament for use in the treatment or prophylaxis of oncogenic viruses, including adenoviruses, retroviruses, and viruses from the papovavirus family, including polyoma viruses and papilloma viruses.

27. Use of a compound according to claim 26 in the manufacture of a medicament for use in the treatment or prophylaxis of conditions or disorders due to HPV infection.

28. Use of a compound as in claim 27 wherein the condition or disorder is warts, genital warts, cervical dysplasia, recurrent respiratory papillomatosis, or cancers associated with papillomavirus infection.

29. A compound selected from the group consisting of:
Methyl 6-chloro-1-(4-methylphenyl)-1,3,4,9-tetrahydro-2H-β-carboline-2-carboxylate;
6-Chloro-1-(4-methylphenyl)-2-(3-phenylpropanoyl)-2,3,4,9-tetrahydro-1H-β-carboline;
Phenylmethyl 6-chloro-1-(4-methylphenyl)-1,3,4,9-tetrahydro-2H-β-carboline-2-carboxylate;
Methyl 6-fluoro-1-(4-methylphenyl)-1,3,4,9-tetrahydro-2H-β-carboline-2-carboxylate;
6-Fluoro-1-(4-methylphenyl)-2-(3-phenylpropanoyl)-2,3,4,9-tetrahydro-1H-3-carboline;
Phenylmethyl 6-fluoro-1-(4-methylphenyl)-1,3,4,9-tetrahydro-2H-β-carboline-2-carboxylate;
Methyl 6-bromo-1-(4-methylphenyl)-1,3,4,9-tetrahydro-2H-β-carboline-2-carboxylate;
6-Bromo-1-(4-methylphenyl)-2-(3-phenylpropanoyl)-2,3,4,9-tetrahydro-1H-β-carboline;
Phenylmethyl 6-bromo-1-(4-methylphenyl)-1,3,4,9-tetrahydro-2H-β-carboline-2-carboxylate;
(1S)-6-Chloro-1-(4-methylphenyl)-2-(3-phenylpropanoyl)-2,3,4,9-tetrahydro-1H-β-carboline;
1-(4-Methylphenyl)-2-[(phenylmethyl)sulfonyl]-2,3,4,9-tetrahydro-1H-β-carboline;
1-(4-Methylphenyl)-2-(methylsulfonyl)-2,3,4,9-tetrahydro-1H-β-carboline;
(4-{[1-(4-Methylphenyl)-1,3,4,9-tetrahydro-2H-b-carbolin-2-yl]sulfonyl}phenyl)amine;
1-(4-Methylphenyl)-2-(3-phenylpropanoyl)-2,3,4,9-tetrahydro-1H-β-carboline;
1-(4-Methylphenyl)-2-(3-phenyl-2-propynoyl)-2,3,4,9-tetrahydro-1H-β-carboline;
1-(4-Methylphenyl)-2-[3-(3-pyridinyl)propanoyl]-2,3,4,9-tetrahydro-1H-β-carboline;
Phenylmethyl {2-[1-(4-methylphenyl)-1,3,4,9-tetrahydro-2H-β-carbolin-2-yl]-2-oxoethyl}carbamate;
6-(Methyloxy)-1-(4-methylphenyl)-2-(3-phenylpropanoyl)-2,3,4,9-tetrahydro-1H-β-carboline;
8-Methyl-1-(4-methylphenyl)-2-(3-phenylpropanoyl)-2,3,4,9-tetrahydro-1H-β-carboline;
6-Methyl-1-(4-methylphenyl)-2-(3-phenylpropanoyl)-2,3,4,9-tetrahydro-1H-β-carboline;
7-Fluoro-1-(4-methylphenyl)-2-(3-phenylpropanoyl)-2,3,4,9-tetrahydro-1H-β-carboline;
7-(Methyloxy)-1-(4-methylphenyl)-2-(3-phenylpropanoyl)-2,3,4,9-tetrahydro-1H-β-carboline; and pharmaceutically acceptable salts and solvates thereof.

30. A compound selected from the group consisting of:
Methyl 6-chloro-1-(4-methylphenyl)-1,3,4,9-tetrahydro-2H-β-carboline-2-carboxylate;
6-Chloro-1-(4-methylphenyl)-2-(3-phenylpropanoyl)-2,3,4,9-tetrahydro-1H-β-carboline;
Phenylmethyl 6-chloro-1-(4-methylphenyl)-1,3,4,9-tetrahydro-2H-β-carboline-2-carboxylate;
Methyl 6-fluoro-1-(4-methylphenyl)-1,3,4,9-tetrahydro-2H-β-carboline-2-carboxylate;
6-Fluoro-1-(4-methylphenyl)-2-(3-phenylpropanoyl)-2,3,4,9-tetrahydro-1H-β-carboline;
Phenylmethyl 6-fluoro-1-(4-methylphenyl)-1,3,4,9-tetrahydro-2H-β-carboline-2-carboxylate;
Methyl 6-bromo-1-(4-methylphenyl)-1,3,4,9-tetrahydro-2H-β-carboline-2-carboxylate;
6-Bromo-1-(4-methylphenyl)-2-(3-phenylpropanoyl)-2,3,4,9-tetrahydro-1H-β-carboline;
Phenylmethyl 6-bromo-1-(4-methylphenyl)-1,3,4,9-tetrahydro-2H-β-carboline-2-carboxylate;
(1S)-6-Chloro-1-(4-methylphenyl)-2-(3-phenylpropanoyl)-2,3,4,9-tetrahydro-1H-β-carboline;
6-(Methyloxy)-1-(4-methylphenyl)-2-(3-phenylpropanoyl)-2,3,4,9-tetrahydro-1H-β-carboline;
6-Methyl-1-(4-methylphenyl)-2-(3-phenylpropanoyl)-2,3,4,9-tetrahydro-1H-β-carboline; and
pharmaceutically acceptable salts or solvates thereof.

31. A compound selected from the group consisting of:
Methyl 6-chloro-1-(4-methylphenyl)-1,3,4,9-tetrahydro-2H-β-carboline-2-carboxylate;
6-Chloro-1-(4-methylphenyl)-2-(3-phenylpropanoyl)-2,3,4,9-tetrahydro-1H-β-carboline;
Phenylmethyl 6-chloro-1-(4-methylphenyl)-1,3,4,9-tetrahydro-2H-β-carboline-2-carboxylate;
Methyl 6-bromo-1-(4-methylphenyl)-1,3,4,9-tetrahydro-2H-β-carboline-2-carboxylate;
6-Bromo-1-(4-methylphenyl)-2-(3-phenylpropanoyl)-2,3,4,9-tetrahydro-1H-β-carboline;
Phenylmethyl 6-bromo-1-(4-methylphenyl)-1,3,4,9-tetrahydro-2H-β-carboline-2-carboxylate;
(1S)-6-Chloro-1-(4-methylphenyl)-2-(3-phenylpropanoyl)-2,3,4,9-tetrahydro-1H-β-carboline; and pharmaceutically acceptable salts or solvates thereof.

32. A pharmaceutical composition comprising a compound according to claim 29 and a pharmaceutically acceptable carrier.

33. A pharmaceutical composition according to claim 32 in the form of a tablet or capsule.

34. A pharmaceutical composition according to claim 32 in the form of a liquid or suspension.
